(19) United States
(12) Patent Application Publication
(10) Pub. No.: US 2023/0384330 A1
(43) Pub. Date: Nov. 30, 2023

(54) COMPOSITIONS AND METHODS RELATING TO THE IDENTIFICATION AND TREATMENT OF IMMUNOTHROMBOTIC CONDITIONS

(71) Applicants: THE REGENTS OF THE UNIVERSITY OF MICHIGAN, Ann Arbor, MI (US); ICAHN SCHOOL OF MEDICINE AT MOUNT SINAI, New York, NY (US)

(72) Inventors: Sascha Goonewardena, Ann Arbor, MI (US); Robert S. Rosenson, New York, NY (US)

(21) Appl. No.: 18/249,674

(22) PCT Filed: Oct. 22, 2021
(86) PCT No.: PCT/US21/56247

§ 371 Date: Apr. 19, 2023

Related U.S. Application Data
(60) Provisional application No. 63/246,528, filed on Sep. 21, 2021.
(60) Provisional application No. 63/104,926, filed on Oct. 23, 2020.

(51) Int. Cl. G01N 33/86

(52) U.S. Cl. CPC G01N 33/86; G01N 2333/7454

(57) ABSTRACT

The present disclosure provides compositions, kits, and methods relating to the identification and treatment of immunothrombotic conditions. In particular, the present disclosure provides novel compositions and methods for identifying whether a subject suffers from an immunothrombotic condition based levels of expression and activation of Tissue Factor (TF) and other immunothrombotic biomarkers.

### CROSS REFERENCE TO RELATED APPLICATIONS

This application claims priority to and the benefit of U.S. Provisional Patent Application No. 63/104,926 filed Oct. 23, 2020, and U.S. Provisional Patent Application No. 63/246,528 filed Sep. 21, 2021, both of which are incorporated herein by reference in their entireties for all purposes.

### FIELD

The present disclosure provides compositions, kits, and methods relating to the identification and treatment of immunothrombotic conditions. In particular, the present disclosure provides novel compositions and methods for identifying whether a subject suffers from an immunothrombotic condition based levels of expression and activation of Tissue Factor (TF) and other immunothrombotic biomarkers.

### BACKGROUND

Recent studies suggest that immunothrombosis is a physiologic response that works in concert with other effector arms of the innate immune system. The immune system can activate coagulation through several procoagulant pathways. For example, pathogens can extrude polyphosphates which can directly lead to complement activation and other immune responses, which can drive expression and coagulation, such as through activation of immunothrombotic regulators (e.g., Tissue Factor). In the same way that unchecked inflammation can lead to tissue damage, dysfunction in immune-mediated coagulation can result in either pathologic thrombosis or coagulopathy, driving myocardial infarction, stroke, and disseminated intravascular coagulation (DIC).

TF is the molecular governor of the extrinsic coagulation pathway and is the key trigger of cell-mediated immunothromobosis. Stress-induced (e.g., infection) activation of TF works in concert with factor VII (FVII) to activate both factor X (FX) and factor IX (FIX), which then leads to thrombin generation and coagulation. In the absence of stress or infection, TF is not normally found in the circulation. One difficulty in understanding the mechanisms of TF driven immunothrombosis is that TF can exist either in dormant or active forms and can be either free or membrane-bound. For example, mechanistic studies have found that in response to pathogens, TF can be activated both in the vasculature and in circulating innate immune cells (e.g., monocytes). In bacterial infections (e.g., Escherichia or Streptococcus) and viral infections (e.g., Ebola and HIV), TF is induced in THP-1 cells (human monocyte cell model) and in circulating monocytes via NFκB and AP-1 pathways. Additionally, in viral infections, TF may also play a direct role in modulating virus infectivity. Activated TF was shown to increase SARS-CoV-1 infectivity by accelerating virus spike protein activation and binding to host ACE2 receptors. Understanding the mechanisms through which TF mediates both thrombotic sequalae and modulates SARS-CoV-2 infectivity have important implications in understanding the pathobiology of COVID-19 and its clinical sequelae.

### SUMMARY

Embodiments of the present disclosure include methods relating to the identification and treatment of immunothrombotic conditions based on the measurement or detection of various biomarkers. In accordance with these embodiments, the method includes obtaining a blood sample comprising a population of peripheral blood mononuclear cells (PBMCs) from a subject having an immunothrombotic condition, and measuring a total level of Tissue Factor (TF) and a level of TF activity in the sample obtained from the subject.

In some embodiments, the method further includes isolating the population of PBMCs from the blood sample. In some embodiments, the method further includes isolating a population of monocytes and/or macrophages from the PBMCs. In some embodiments, the method includes measuring the total TF level and the TF activity level from the population of monocytes and/or macrophages.

In some embodiments, the method includes measuring total TF levels by performing an immunoassay. In some embodiments, the method includes measuring total TF levels by performing a fluorometric assay. In some embodiments, measuring TF activity level comprises measuring Factor Xa.

In some embodiments, the method further includes determining a ratio of total TF levels to TF activity levels.

In some embodiments, the method further includes obtaining a total TF level and a TF activity level in a sample obtained from a control subject. In some embodiments, the method further comprises measuring a total TF level and a TF activity level in a sample obtained from a control subject. In some embodiments, the TF activity level is elevated in the sample from the subject having an immunothrombotic condition as compared to the TF activity level in the control.

In accordance with these embodiments, the immunothrombotic condition identified and/or treated with the compositions and methods of the present disclosure can be any of a virus infection, atherosclerotic cardiovascular disease (ASCVD), coronary heart disease (CHD), rheumatoid arthritis (RA), inflammatory bowel disease (IBD), chronic kidney disease, and any other acute and/or chronic inflammatory disorder. In some embodiments, the virus infection is a SARS-CoV-2 infection. In some embodiments, the IBD is Crohn's disease.

In some embodiments, the immunothrombotic condition is characterized by an altered level of at least one biomarker. In some embodiments, the at least one biomarker comprises hyaluronan (Hyal), syndecan-1 (SDC1), interleukin 6 (IL-6), tumor necrosis factor alpha (TNFα), Lipoprotein(a) (Lp(a)), interleukin 8 (IL-8), P-selectin glycoprotein ligand-1 (PSGL-1), and oncostatin M (OSM), heparan sulfate (HS), high-sensitivity cardiac troponin hs-cTn), high-sensitivity C-reactive protein (hs-CRP), low-density lipoprotein (LDL), von Willebrand factor (vWF), and any combinations thereof.

In some embodiments, the method further includes measuring a level of at least one biomarker. In some embodiments, the at least one biomarker comprises hyaluronan (Hyal), syndecan-1 (SDC1), interleukin 6 (IL-6), tumor necrosis factor alpha (TNFα), Lipoprotein(a) (Lp(a)), interleukin 8 (IL-8), P-selectin glycoprotein ligand-1 (PSGL-1), and oncostatin M (OSM), and any combinations thereof. In some embodiments, the at least one biomarker is Lp(a), and wherein the Lp(a) is elevated in the sample from the subject having an immunothrombotic condition as compared to the Lp(a) level in the control. In some embodiments, the at least one biomarker is IL-6, and wherein the IL-6 is elevated in the sample from the subject having an immunothrombotic condition as compared to the IL-6 level in the control. In some embodiments, the method includes measuring at least one additional biomarker selected from the group consisting of RAGE, CD40, CCL25, CXCL6, TNFα, CXCL5, PD-L1, MMP1, IL-18, CXCL1, Trail, OSM, uPA, IL-7, IL-8, Dkk-1, CCL17, IL-18, LOX1, CXCL1, PAR1, Angpt1, and CD40L. In some embodiments, the at least one biomarker is altered in the sample from the subject having an immunothrombotic condition as compared to the level in the control.

In some embodiments, the method further comprises treating the subject based on the TF activity level. In some embodiments, treating the subject comprises administering an anti-thrombotic therapy. In some embodiments, the anti-thrombotic therapy comprises administering a composition comprising heparin and/or Annexin V.

In some embodiments, treating the subject comprises administering an apoptotic modulator. In some embodiments, the apoptotic modulator induces apoptosis and treats the subject. In some embodiments, the apoptotic modulator reduces apoptosis and treats the subject.

Embodiments of the present disclosure also include a method for treating a subject having or suspected of having an immunothrombotic condition. In accordance with these embodiments, the method includes obtaining a blood sample comprising a population of peripheral blood mononuclear cells (PBMCs) from a subject having an immunothrombotic condition, measuring a total level of Tissue Factor (TF) and a level of TF activity in the sample obtained from a subject, and administering anti-thrombotic therapy and/or an apoptotic modulator to the subject to treat the immunothrombotic condition.

In some embodiments, the method further comprises isolating the population of PBMCs from the blood sample. In some embodiments, measuring total TF levels comprises performing an immunoassay. In some embodiments, measuring total TF levels comprises performing a fluorometric assay. In some embodiments, measuring the TF activity level comprises measuring Factor Xa.

In some embodiments, the method further comprises obtaining a total TF level and a TF activity level in a sample obtained from a control subject. In some embodiments, the TF activity level is elevated in the sample from the subject having an immunothrombotic condition as compared to the TF activity level in the control. In some embodiments the immunothrombotic condition is selected from the group consisting of a virus infection, atherosclerotic cardiovascular disease (ASCVD), coronary heart disease (CHD), rheumatoid arthritis (RA), inflammatory bowel disease (IBD), chronic kidney disease, and any other acute and/or chronic inflammatory disorder. In some embodiments the virus infection is a SARS-CoV-2 infection.

In some embodiments the anti-thrombotic therapy and/or the apoptotic modulator is administered to the subject based on the TF activity level. In some embodiments, the anti-thrombotic therapy comprises administering a composition comprising heparin and/or Annexin V. In some embodiments, the apoptotic modulator induces apoptosis and treats the subject. In some embodiments the apoptotic modulator reduces apoptosis and treats the subject.

Embodiments of the present disclosure also include a kit comprising a Tissue Factor (TF) detection agent, a TF activity detection agent, and instructions for performing an assay to determine a ratio of total TF to activated TF in a blood sample comprising a population of peripheral blood mononuclear cells (PBMCs).

### BRIEF DESCRIPTION OF THE DRAWINGS

FIG. 1: Circulating TF is increased in hospitalized COVID-19 patients compared with HC. Plasma samples were collected from 38 consecutive patients on admission with PCR-confirmed SARS-CoV-2. Plasma was obtained from 4 healthy controls (HC). Plasma concentrations are expressed as mean±SD. *p<0.001.

FIGS. 2A-2B: Monocytes activated by inflammatory stimuli increase TF/CD142 expression and activity. PBMC were isolated from healthy controls and stimulated with LPS (1 ug/ml for 4 h) and compared with no treatment (NT) controls. A) Flow cytometric analysis demonstrating rapid and robust induction of CD142 in LPS treated CD14+ monocytes (blue) compared with NT CD14+ monocytes (red). B) Tissue factor activity was assessed in both cells and supernatants in PBMC treated with LPS (1 ug/ml for 4 h). The tissue factor activity was quantified based on the ability of TF/FVIIa to activate FX to factor Xa. The amidolytic activity of the TF/FVIIa complex is quantitated by the amount of FXa produced using a highly specific FXa substrate which releases a chromophore upon cleavage. *p<0.05.

FIGS. 3A-3B: Heparin augments type I IFN/STAT1 signaling and tends to blunt CD142/TF transcriptional induction in response to LPS. A) CD14+ monocytes were stimulated with LPS (1 ug/ml; 4 h) and heparin (Hep) (10 u/ml) and signal pathway activation was probed using microfluidic proteomics. There were no differences in p-p65 and p-PKC activation between groups. However, phospo-STAT1 activation was augmented in monocytes cotreated with LPS and Hep. Normalized activation determined by normalizing phospho proteins to total protein. B) qRT-PCR of CD14+ monocytes treated with LPS with and without Hep. CD142/TF is induced in response to LPS and there is a trend to blunted transcriptional expression in monocytes cotreated with LPS and Hep. Fold-change determined normalizing to GAPDH and to NT groups. *p<0.05; #p=0.07

FIGS. 4A-4C: Recovered COVID-19 patients have increased CD142/TF expression on monocytes compared with HC. A) Representative biaxial gating strategy to identify monocyte populations using mass cytometry. B) viSNE map based on CD142/TF marker channel; 30 surface markers profiled (customized Fluidigm human panel). C) Increased CD14+ monocytes in COVID-19 patients compared with HC and also an increase in CD142/TF expression on circulating monocytes from recovered COVID-19 patients compared with HC. n=8 recovered COVID-19 patients; n=3 HC. Cell populations are expressed as mean±SD. *p<0.05.

FIGS. 5A-5C: Annexin V peptide pretreatment blocks LPS induction of CD142/Tissue factor and CD142/Tissue factor activity. PBMC were pre-treated for 1 hr with Annexin V (10 ug/ml) or PBS and then stimulated with LPS (1 ug/ml) for 4 hours. PBMC were analyzed with either flow cytometry or tissue factor activity assay. A) Representative SSC vs. FSC gating strategy for Monocytes and Lymphocytes; B) CD142/Tissue factor was assessed by flow cytometry. Annexin V pre-treatment blocks CD142/Tissue factor induction by LPS. C). CD142/Tissue factor activity using substrate cleavage and FXa generation. Results are representative of three independent experiments. *p<0.05

FIG. 6: PBMC from COVID-19 patients have higher TF activity compared with healthy controls (HC). PBMC were isolated from HC and COVID-19 patients after index hospitalization. Tissue factor activity was assessed in PBMC. The tissue factor activity was quantified based on the ability of TF/FVIIa to activate FX to factor Xa. The amidolytic activity of the TF/FVIIa complex is quantitated by the amount of FXa produced using a FXa substrate which releases a chromophore upon cleavage.

FIGS. 7A-7B: Representative data indicating Toll-like receptor ligands induce monocyte tissue factor expression and monocytes from critical COVID-19 patients express TF.

FIGS. 8A-8C: Representative data illustrating proteomic signatures in hospitalized COVID-19 patients.

FIGS. 9A-9D: Representative data from immune stress tests indicating immunoparalysis in critical COVID-19 patients.

FIG. 10: Lp(a) increases NFκB induction on THP1-Dual monocytes; addition of TLR2 decreases activation effect of Lp(a). THP1-Dual monocytes stimulated for 24 h with 77.5 ug/mL Lp(a), 77.5 μg/mL Lp(a)+10 μg/mL TR2, 77.5 μg/mL Lp(a)+10 μg/mL TR4, and 100 ng/mL Pam2CSK4. After 24 h stimulation, supernatant was collected and NFκB activation was assessed by measuring the levels of SEAP using QUANTI-Blue assay. Levels of SEAP were determined by reading the optical density at 640 nm. *p-value<0.0001 (ANOVA).

FIGS. 11A-11D: Higher Lp(a) concentrations increase CHD risk, and ASCVD subjects with elevated Lp(a) have increased circulating vascular and inflammatory markers. A) Cumulative incidence of CHD events by Lp(a) levels among REGARDS study participants (n=1948) on a statin with history of ASCVD. Higher Lp(a) concentration is a risk factor for CHD events, but in both White and Black participants. To prospectively examine the effects of Lp(a) on the plasma proteome, plasma was isolated from ASCVD subjects with an elevated Lp(a) (8) and age-matched controls (11). Proteomic signatures were determined using PEA of 184 plasma markers. B) Heatmap of 25 samples (rows) and 184 protein markers demonstrating clustering of ASCVD subjects with high Lp(a) compared to age-matched controls. C) Scatterplot of Lp(a) samples and age-matched controls along two principal components. D) Volcano plot comparing plasma proteome from Lp(a) subjects to age-matched controls. 54 out of 184 proteins have statistically significant difference in means between the two groups including IL-8, Onchostatin M (OSM), lectin-like oxidized LDL receptor-1 (LOX-1), and urokinase-type plasminogen activator (uPA).

FIGS. 12A-12C: Inflammatory mediators increase CD142/TF expression and activity in monocytes. A) PBMC were isolated from HC and stimulated with Pam2CSK4(TLR2), Poly(I:C) (TLR3), and LPS (TLR4) for 4 h. Samples were barcoded and stained (24 surface markers), and mass cytometry was performed. viSNE plots demonstrate increased CD142/TF expression compared to no treated (NT) controls. Circles represent CD14+ monocytes. B) PBMC were isolated and stimulated with LPS and Ang II (vascular activator) and TF activity was quantified based on the ability of TF/FVIIa to activate FX to factor Xa. The amidolytic activity of the TF/FVIIa complex is quantitated by the amount of FXa produced using a FXa substrate which releases a chromophore upon cleavage. *p<0.05. C) To probe for the mechanisms that regulate TF expression in circulating monocytes, PBMC were isolated and stimulated with LPS with or without Annexin V (AnnV) to block apoptotic sensing (through masking of PS on target cells/particles) in monocytes. Intriguingly, blocking PS sensing with AnnV greatly reduced CD142/TF induction in response to LPS. *p<0.05. Results representative of 3 independent experiments.

FIGS. 13A-13C: Bulk transcriptomics (using RNA-seq) and bioinformatic analysis of circulating PBMCs from subjects with CAD and low Lp(a) (N=3) and subjects with CAD and high Lp(a) (N=6). A) Heat map depicting the top 50 differentially expressed genes in PBMC between low and high Lp(a) subjects. B) Volcano plot with circles representing differentially expressed (DE) genes between high Lp(a) and low Lp(a) subjects. Horizontal axis is the log fold change, and the vertical axis is the negative base-10 logarithm of the p-value. Upregulated (red) and downregulated (blue) DE genes are denoted by circles. DE thresholds; fold change: 0.2; p-value: 0.05. C) Pathway analysis of differentially regulated biological pathways between high Lp(a) and low Lp(a) subjects. Dots representing pathways are positioned by their p-values from two different analyses: an impact analysis measuring total perturbation accumulation vs. a classical overrepresentation analysis. Pathways with significant combined P-values are depicted in red. The size of each dot denotes the total number of genes in the corresponding pathway. Yellow dot represents antigen processing and presentation pathway.

FIGS. 14A-14D: CD142 expression is upregulated in response to both LPS and LpA in monocytes and granulocytes. PBMCs were isolated and treated with 1 ug/mL LPS, 155 ug/mL LpA, or a combination of the above for 4 hours with gentle vortexing every 30 minutes. Cells were blocked with Tru-Stain FcX for 10 minutes and then treated with human anti CD142-PE for 30 minutes on ice. Cells were then analyzed via flow cytometry. (A) Representative FSC vs. SSC gating strategy. (B) Percent positive CD142+ for lymphocytes. (C) Percent positive CD142+ for monocytes. (D) Percent positive CD142+ for granulocytes. N=3. *p-value<0.0001 (ANOVA).

FIGS. 15A-15B: (A) Gating strategy employed to identify CD142+ subpopulations in monocytes and lymphocytes in human PBMCs (shown for NT). The data was gated for DNA intercalators and CD45+ population and then de-barcoded as individual FCS files. (B) Data demonstrating significantly higher Lp(a) levels in these cells.

### DETAILED DESCRIPTION

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

“Correlated to” as used herein refers to compared to.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

An “absolute amount” as used herein refers to the absolute value of a change or difference between at least two assay results taken or sampled at different time points and, which similar to a reference level, has been linked or is associated herein with various clinical parameters (e.g., presence of disease, stage of disease, severity of disease, progression, non-progression, or improvement of disease, etc.). “Absolute value” as used herein refers to the magnitude of a real number (such as, for example, the difference between two compared levels (such as levels taken at a first time point and levels taken at a second time point)) without regard to its sign, i.e. regardless of whether it is positive or negative.

“Antibody” and “antibodies” as used herein refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25 (11):1290-1297 (2007) and PCT International Application WO 2001/058956, the contents of each of which are herein incorporated by reference), or domain antibodies (dAbs) (e.g., such as described in Holt et al. (2014) Trends in Biotechnology 21:484-490), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site Immunoglobulin molecules can be of any type (for example, IgG, IgE, IgM, IgD, IgA, and IgY), class (for example, IgG1, IgG2, IgG3, IgG4, IgA1, and IgA2).

“Antibody fragment” as used herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Coefficient of variation” (CV), also known as “relative variability,” is equal to the standard deviation of a distribution divided by its mean.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, whole blood, serum or plasma sample, in accordance with the methods described herein and other methods known in the art. Some components can be in solution or lyophilized for reconstitution for use in an assay.

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like).

“Dynamic range” as used herein refers to range over which an assay readout is proportional to the amount of target molecule or analyte in the sample being analyzed. The dynamic range can be the range of linearity of the standard curve.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Fragment,” “biomarker fragment,” or “biomarker peptide” as used herein includes any identifying fragment of any of the biomarkers identified and described herein. “Fragment(s)” include nucleic acids, polynucleotides, peptides, prototypic peptides, proteolytic peptides, isoforms, including SNPs or post-translationally modified forms, and any endogenously or exogenously induced forms, of any biomarker identified and described herein.

“Isolated polynucleotide” as used herein includes a polynucleotide (e.g., of genomic, cDNA, or synthetic origin, or a combination thereof) that, by virtue of its origin is not associated with all or a portion of a polynucleotide with which the “isolated polynucleotide” is found in nature, is operably linked to a polynucleotide that it is not linked to in nature, and/or does not occur in nature as part of a larger sequence.

“Limit of Blank (LoB)” as used herein refers to the highest apparent analyte concentration expected to be found when replicates of a blank sample containing no analyte are tested.

“Limit of Detection (LoD)” as used herein refers to the lowest concentration of the measurand (i.e. a quantity intended to be measured) that can be detected at a specified level of confidence. The level of confidence is typically 95%, with a 5% likelihood of a false negative measurement. LoD is the lowest analyte concentration likely to be reliably distinguished from the LoB and at which detection is feasible. LoD can be determined by utilizing both the measured LoB and test replicates of a sample known to contain a low concentration of analyte. The LoD term used herein is based on the definition from Clinical and Laboratory Standards Institute (CLSI) protocol EP17-A2 (“Protocols for Determination of Limits of Detection and Limits of Quantitation; Approved Guideline—Second Edition,” EP17A2E, by James F. Pierson-Perry et al., Clinical and Laboratory Standards Institute, Jun. 1, 2012).

“Limit of Quantitation (LoQ)” as used herein refers to the lowest concentration at which the analyte can not only be reliably detected but at which some predefined goals for bias and imprecision are met. The LoQ may be equivalent to the LoD or it could be at a much higher concentration.

“Linearity” refers to how well the method or assay's actual performance across a specified operating range approximates a straight line. Linearity can be measured in terms of a deviation, or non-linearity, from an ideal straight line. “Deviations from linearity” can be expressed in terms of percent of full scale. In some of the methods disclosed herein, less than 10% deviation from linearity (DL) is achieved over the dynamic range of the assay. “Linear” means that there is less than or equal to about 20%, about 19%, about 18%, about 17%, about 16%, about 15%, about 14%, about 13%, about 12%, about 11%, about 10%, about 9%, or about 8% variation for or over an exemplary range or value recited.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). However, it is well-known that reference levels may vary depending on the nature of the assay used and that assays can be compared and standardized. It further is well within the ordinary skill of one in the art to adapt the disclosure herein for other assays to obtain specific reference levels for those other assays based on the description provided by this disclosure. Whereas the precise value of the reference level may vary between assays, the findings as described herein should be generally applicable and capable of being extrapolated to other assays.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,” “test sample,” “specimen,” “sample from a subject,” and “patient sample” as used herein may be used interchangeably and may be a sample of blood, such as whole blood, tissue, skin, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal and a human. In some embodiments, the subject may be a human or a non-human. The subject or patient may be undergoing other forms of treatment.

“Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

The terms “administration of” and “administering” a composition as used herein refers to providing a composition of the present disclosure to a subject in need of treatment. The compositions of the present disclosure may be administered by topical (e.g., in contact with skin or surface of body cavity), oral, parenteral (e.g., intramuscular, intraperitoneal, intravenous, ICV, intracisternal injection or infusion, subcutaneous injection, or implant), by spray, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier and/or excipient. When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

The term “pharmaceutical composition” as used herein refers to a composition that can be administered to a subject to treat or prevent a disease or pathological condition, and/or to improve/enhance one or more aspects of a subject's physical health. The compositions can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and more particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

As used herein, the term “effective amount” generally means that amount of a drug or pharmaceutical agent that will elicit the biological or medical response of a tissue, system, animal or human that is being sought, for instance, by a researcher or clinician. Furthermore, the term “therapeutically effective amount” generally means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “combination” and derivatives thereof, as used herein, generally means either, simultaneous administration or any manner of separate sequential administration of a therapeutically effective amount of Compound A, or a pharmaceutically acceptable salt thereof, and Compound B or a pharmaceutically acceptable salt thereof, in the same composition or different compositions. If the administration is not simultaneous, the compounds are administered in a close time proximity to each other. Furthermore, it does not matter if the compounds are administered in the same dosage form.

As used herein, the term “salts” and “pharmaceutically acceptable salts” generally refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids can include, e.g., acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethanedisulfonic, oxalic, and isethionic, and the like. Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, isopropanol, and the like. Lists of suitable salts can be found, for example, in Remington's Pharmaceutical Sciences, 17th ed., Mack Publishing Company, Easton, Pa., 985.

### 2. Tissue Factor

Embodiments of the present disclosure include the finding that circulating TF is elevated in patients with COVID-19 compared with controls (e.g., healthy controls). Additionally, it was found that patients who have recovered from SARS-CoV-2 infections have alterations in circulating monocyte populations and increased monocyte TF expression, suggesting that they have residual immunothrombotic risk. Because of the associations between thrombosis and SARS-CoV-2 infections, experiments were conducted investigate further the use of TF as a biomarker for immunothrombotic conditions.

Vascular and immune dysfunction are hallmarks of severe acute respiratory syndrome coronavirus-2 (SARS-CoV-2) infections and coronavirus disease 2019 (COVID-19). The leading cause of death in patients with COVID-19 is hypoxic respiratory failure secondary to acute respiratory distress syndrome (ARDS). A key pathogenic driver underlying COVID-19 is infection of airway cells, resulting in excessive inflammation and vascular dysfunction. Thrombosis is a major cause of morbidity and mortality in COVID-19 patients. In contrast to more vascular-driven thrombotic states, clinical trials of therapeutic anticoagulation in COVID-19 have had mixed results, with the clinical benefits mostly restricted to patients with mild and moderate disease with little evidence of benefit in the most severe patients. Although the understanding of the immunologic drivers of COVID-19 has rapidly evolved, much less is known about the mechanisms mediating immune-driven thrombosis in COVID-19. To further understand the mechanisms linking vascular and immune dysfunction in COVID-19, 192 vascular and inflammatory markers were measured in a hospitalized cohort of moderate, severe, and critical COVID-19 patients and 10 healthy controls (HC).

Unchecked inflammation and thrombosis, hallmarks of COVID-19, drive much of the morbidity and mortality of SARS Coronavirus-19 (SARS-CoV-2) infections. Myeloid cells (e.g., Mϕ) are central in orchestrating the innate immune response to contain and eliminate the virus, and coordinate tissue preservation and repair. Severe SARS-CoV-2 infection can trigger host cell death, endothelial dysfunction, and profound activation of platelets and circulating immune cells. Although monocytes and monocyte-derived macrophages lie at the nexus of inflammation and thrombosis in COVID-19, little is known about thromboinflammatory mechanisms that converge at the tripping point between protective anti-viral defense and devastating hyperinflammation and pathologic thrombosis.

Coronavirus disease 2019 (COVID-19) is caused by the beta-coronavirus severe acute respiratory syndrome coronavirus-2 (SARS-CoV-2). SARS-CoV-2 enters host cells following fusion of the virus spike (S) surface protein with host angiotensin-converting enzyme 2 (ACE2), a type I transmembrane metallocarboxypeptidase expressed broadly, especially in the respiratory tract. Intracellular viral replication induces apoptosis and triggers the release of inflammatory cytokines (IL6 and TNFα). Circulating inflammatory and vasoactive molecules can induce Mϕ (macrophage) tissue factor (TF) expression, which potently activates the extrinsic coagulation cascade, culminating in the generation of thrombin, a key molecular executioner of intravascular thrombosis.

The leading cause of death in patients with COVID-19 is hypoxic respiratory failure secondary to acute respiratory distress syndrome (ARDS). COVID-19 presents with a spectrum of clinical phenotypes, with most patients exhibiting either mild or moderate symptoms. However, approximately 15% of patients progress to more severe disease necessitating hospitalization and cardiopulmonary support. Current epidemiological data suggests that COVID-19 has a mortality rate several times greater than that of seasonal influenza. Additionally, elderly patients and patients with underlying comorbidities such as cardiovascular disease, diabetes mellitus, chronic lung disease, chronic kidney disease, obesity, and cancer have a higher risk of COVID-19 complications and an increased mortality rate compared with infected young, healthy adults. Circulating inflammatory and vascular markers have been associated with more severe SARS-CoV-2 infections (troponin, D-dimer, lymphocyte counts, and some inflammatory cytokines), suggesting a mechanistic link between vascular and immune dysfunction in COVID-19.

Multi-organ damage in COVID-19 is related to unchecked inflammation and direct viral-induced organ and cell dysfunction. SARS-CoV-2 infects the host through interactions with the angiotensin converting enzyme 2 (ACE2) receptor. The ACE2 receptor is expressed in the lung, heart, kidney, and intestine. ACE2 receptors are also expressed by endothelial cells (EC). Whether vascular derangements in COVID-19 are due to viral infection of EC or immune-related pathology (or some combination of the two) in response to the virus remains unknown. To date, EC have been largely overlooked as a therapeutic target in COVID-19; emerging evidence suggests that these cells contribute to the initiation and propagation of ARDS in COVID-19 by altering vascular integrity, promoting micro- and macrovascular thrombosis, and inducing vascular inflammation. A mechanistic understanding of the direct and indirect SARS-CoV-2 effects on the vasculature is critical and will help to clarify vascular-immune interactions that underlie the pathobiology of COVID-19.

As knowledge of COVID-19 has evolved, it is clear that vascular and thrombotic complications are common in COVID-19. A recent study found evidence of direct viral-mediated dysfunction of the vascular endothelium in a series of patients suffering from severe COVID-19. Additionally, in a small cohort of COVID-19 patients, a previous study found that COVID-19 patients had striking reductions in microvascular density and had evidence of GAC damage providing direct clinical evidence of vascular dysfunction. The endothelial GAC is comprised of proteoglycans, glycosaminoglycan (GAG) chains, and glycoproteins. Syndecan-1 (SDC1), a canonical proteoglycan, helps maintain vascular integrity and regulates endothelial responses. GAG chains that bind to proteoglycans include chondroitin sulfate and heparan sulfate, some of which have been implicated in SARS-CoV-2 infectivity. In contrast to GAGs, hyaluronan (Hyal) is a linear, neutral molecule that interacts cell-membrane CD44 and can form complexes with other GAGs, complexes that together stabilize the GAC. In sepsis, the GAC can be actively degraded by enzymes including metalloproteinases, heparanase, and hyaluronidase Immune-mediated GAC degradation increases vascular permeability, microvascular thrombosis, and leukocyte recruitment. Observational studies in sepsis populations have found an association between circulating levels of GAC degradation products and end-organ dysfunction and mortality.

Similar to the endothelial GAC disruption in sepsis-mediated ARDS, it was hypothesized that endothelial GAC disruption is associated with vascular dysfunction in SARS-CoV-2 infections and precedes the genesis of ARDS in COVID-19. Therefore, as described further herein, experiments were conducted to evaluate if GAC components and inflammatory biomarkers were elevated in hospitalized COVID-19 patients compared with healthy controls (HC) and if these markers correlated with disease severity. Results demonstrated that Hyal and SDC1 were increased in COVID-19 patients and these markers correlated with disease severity. These results build on prior studies and further reveals an important intersection between GAC remodeling and SARS-CoV-2 infections, an intersection which could have diagnostic and therapeutic implications and furthers understanding of the pathobiology of COVID-19.

In hospitalized COVID-19 patients, data demonstrated that plasma TF is increased compared with healthy controls (HC). Additionally, it was found that convalescent COVID-19 patients have increased Mϕ and Mϕ-TF expression compared to age-matched controls, suggesting that even in the recovery phase, COVID-19 patients have heightened thrombotic risk. Mechanistically, data demonstrated that toll-like receptor (TLR) ligands rapidly induce TF expression in CD14+Mϕ from peripheral blood mononuclear cells (PBMC). Interestingly, when recognition of phosphatidylserine (PS), an apoptotic marker, is pharmacologically masked, TLR-driven TF expression is reduced, suggesting that parallel Mϕ sensing of TLR ligands and apoptotic cells/bodies drives immunothrombosis in COVID-19.

Therefore, one objective of the present disclosure was to define the molecular mechanisms that govern heparin effects on monocyte activation and myeloid TF-mediated thrombosis in PBMCs. Results suggest the development of a maladapted immune response profile associated with severe COVID-19 outcome and early immune signatures that correlate with divergent disease trajectories.

In accordance with the above, embodiments of the present disclosure include methods relating to the identification and treatment of immunothrombotic conditions (e.g., COVID-19) based on the measurement and/or detection of various biomarkers. In some embodiments, the methods include obtaining a blood sample comprising a population of peripheral blood mononuclear cells (PBMCs) from a subject having an immunothrombotic condition, and measuring a total level of Tissue Factor (TF) and a level of TF activity in the sample obtained from the subject. As described further herein, analysis of TF activity relative to total TF levels provides a robust and accurate means for assessing immunothrombotic risk in a subject, which has previously been unrecognized.

In some embodiments, the method includes isolating a population of PBMCs from a blood sample. In some embodiments, the method further includes isolating a population of monocytes and/or macrophages from the PBMCs. In some embodiments, the method includes measuring the total TF level and the TF activity level from the population of monocytes and/or macrophages. As would be recognized by one of ordinary skill in the art based on the present disclosure, methods for isolating PBMCs from a blood sample can be performed using a variety of methods, including, but not limited to the methods described herein. In some embodiments, such methods include isolating CD14+ and CD14+/CD142+ monocytes.

Assessing total TF levels and TF activity levels can be performed using various means known in the art, including but not limited to, immunoassays, biochemical assays, enzymatic assays, fluorometric assay, and the like. In some embodiments, methods for assessing a subject for immunothrombotic risk includes measuring total TF levels by performing an immunoassay. In other embodiments, the method includes measuring total TF levels by performing a fluorometric assay. In some embodiments, measuring TF activity level comprises measuring Factor Xa. As described further herein, TF can be quantified based on the ability of TF/FVIIa to activate FX to factor Xa. The amidolytic activity of the TF/FVIIa complex can be measured and/or quantitated by the amount of FXa produced using a highly specific FXa substrate which releases a chromophore upon cleavage.

In some embodiments, the method further includes determining a ratio of total TF levels to TF activity levels. In some embodiments, the method includes assessing a total TF and/or TF activity based on absolute measurements, and then comparing these absolute values. In some embodiments, the method includes assessing a total TF and/or TF activity based on relative measurements, and then comparing these relative values. In some embodiments, the method includes assessing a total TF and/or TF activity based, and then comparing these values to reference values to determine immunothrombotic risk.

In some embodiments, the method further includes obtaining a total TF level and a TF activity level in a sample obtained from a control subject, and assessing or comparing the control sample to that of a subject sample. As would be recognized by one of ordinary skill in the art based on the present disclosure, a control sample can include, but is not limited to, a sample obtained from a healthy subject, a sample obtained from a subject being treated with one or more medications for treating an immunothrombotic condition, a sample obtained from a subject with an immunothrombotic condition, and/or a sample obtained from a subject having a certain immunothrombotic status (e.g., genetic or proteomic profile). In some embodiments, obtaining a total TF level and a TF activity level from a control sample can include directly measuring these levels from a blood sample obtained from the control subject. In other embodiments, obtaining a total TF level and a TF activity level from a control sample can include consulting a reference value (e.g., lookup table) that corresponds to aggregate total TF and TF activity values for a given set of control samples.

In some embodiments, the TF activity level is elevated in the sample from the subject having an immunothrombotic condition as compared to the TF activity level in the control. As would be recognized by one of ordinary skill in the art based on the present disclosure, elevated TF activity levels in a subject can be determined with reference to total TF levels. Additionally, the degree or amount of elevated TF activity levels in a subject sample can be determined based on one or more control samples (e.g., total TF levels and TF activity levels obtained from one or more control samples). In some embodiments, the degree or amount of elevated TF activity levels in a subject is indicative of an immunothrombotic condition. For example, TF activity levels in a subject sample can be elevated by any degree or amount that represents a statistically significant difference from a control sample.

In accordance with these embodiments, an immunothrombotic condition identified and/or treated with the compositions and methods of the present disclosure can be any currently recognized disease or condition that includes one or more characteristics (e.g., symptoms) of a thrombotic and/or immune disease or condition, as well as any disease or condition yet to be identified that includes one or more characteristics of a thrombotic and/or immune disease or condition. For example, an immunothrombotic condition that can be identified and/or treated with the compositions and methods of the present disclosure includes, but is not limited to, a virus infection, atherosclerotic cardiovascular disease (ASCVD), coronary heart disease (CHD), rheumatoid arthritis (RA), inflammatory bowel disease (IBD), chronic kidney disease, and any other acute and/or chronic inflammatory disorder. In some embodiments, the virus infection is a SARS-CoV-2 infection. In some embodiments, the IBD is Crohn's disease.

In some embodiments, the immunothrombotic condition is characterized by an altered level of at least one biomarker, in addition to elevated TF activity levels. In some embodiments, the biomarker includes, but is not limited to, hyaluronan (Hyal), syndecan-1 (SDC1), interleukin 6 (IL-6), tumor necrosis factor alpha (TNFα), Lipoprotein(a) (Lp(a)), interleukin 8 (IL-8), P-selectin glycoprotein ligand-1 (PSGL-1), and oncostatin M (OSM), heparan sulfate (HS), high-sensitivity cardiac troponin hs-cTn), high-sensitivity C-reactive protein (hs-CRP), low-density lipoprotein (LDL), von Willebrand factor (vWF), and any combinations thereof. In accordance with these embodiments, the method can include measuring a level of at least one of these biomarkers. The level of the biomarker can be altered in a subject sample as compared to a control sample. In some embodiments, the altered level is an elevated level, and in other embodiments, the altered level is a decreased level. As would be recognized by one of ordinary skill in the art based on the present disclosure, the degree or amount of increase or decrease of the biomarker will depend on various factors, such as the biomarker being measured and the nature of the assay by which it is measured.

In some embodiments, and as described further herein, the biomarker is Lp(a), and the Lp(a) is elevated in the sample from the subject having an immunothrombotic condition as compared to the Lp(a) level in the control. In some embodiments, and as described further herein, the biomarker is IL-6, and the IL-6 is elevated in the sample from the subject having an immunothrombotic condition as compared to the IL-6 level in the control. In some embodiments, the method includes measuring at least one additional biomarker selected from the group consisting of RAGE (UniProtKB Q49A77), CD40 (UniProtKB P29965), CCL25 (UniProtKB 015444), CXCL6 (UniProtKB P80162), TNFα (UniProtKB P01375), CXCL5 (UniProtKB P42830), PD-L1 (UniProtKB Q9NZQ7), MMPI (UniProtKB P03956), IL-18 (UniProtKB Q14116), CXCL1 (UniProtKB P09341), Trail (UniProtKB P50591), OSM (UniProtKB P13725), uPA (UniProtKB P00749), IL-7 (UniProtKB P13232), IL-8 (UniProtKB P10145), Dkk-1 (UniProtKB 094907), CCL17 (UniProtKB Q92583), IL-18 (UniProtKB Q14116), LOX1 (UniProtKB P78380), CXCL1 (UniProtKB P09341), PAR1 (UniProtKB P25116), Angpt1 (UniProtKB Q15389), and/or CD40L (UniProtKB P29965), including any combinations thereof.

Results. Numerous studies have demonstrated that inflammatory markers are elevated in COVID-19 patients (e.g., IL-6 and TNFα) and the data of the present disclosure suggest that thrombotic markers (TF) are elevated in hospitalized COVID-19 patients compared with healthy controls. Additionally, as described further herein, results demonstrated that circulating monocytes from controls respond to inflammatory stimuli and induce surface expression and transcription of TF. To further explore if heparin itself can modulate monocyte-mediated inflammation, monocytes were isolated from controls and treated with LPS and heparin. These data suggest that heparin can augment STAT1 signaling in monocytes, providing a possible mechanism through which heparin itself can increase type I IFN signaling that may be beneficial in the early stages of SARS-CoV-2 infection. Additionally, experiments were conducted to generate an optimized mass cytometry immune marker panel and a profile for circulating peripheral blood monocytes from patients with a history of COVID-19. Results indicated that even in recovered COVID-19 patients, there was evidence of TF+ circulating monocytes compared with controls.

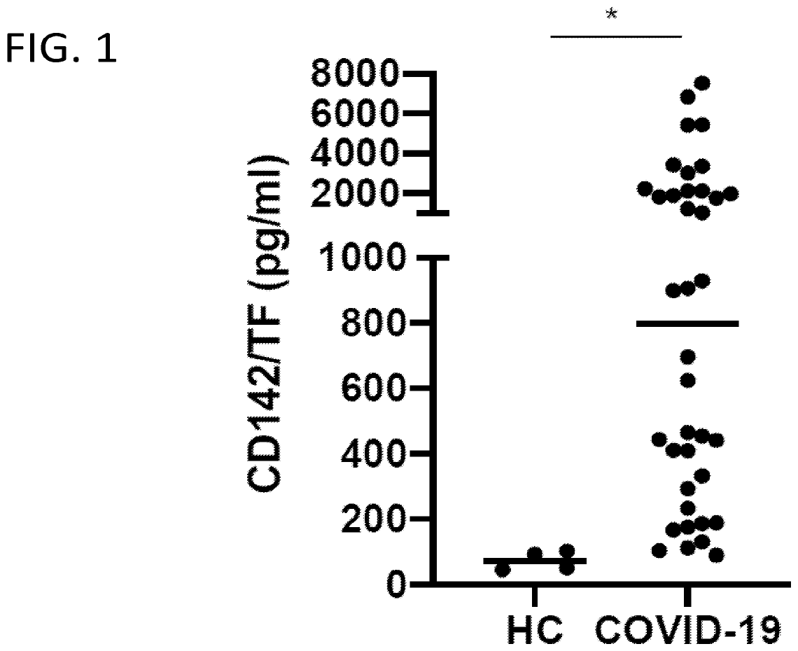

Results of the present disclosure indicated that hospitalized COVID-19 patients have increased circulating TF. The initial coagulopathy of COVID-19 presents with prominent elevation of D-dimer. D-dimer is a protein fragment present in the blood when coagulation has been triggered. Because of the increased thrombosis and elevated D-dimer levels observed in COVID-19 patients, studies were conducted to examine if thrombosis could be driven by extrinsic pathway activation through TF. Using a validated ELISA, circulating TF was profiled in 38 COVID-19 patients within 72 hours of admission and compared them to 4 HC. Results demonstrated striking elevations in circulating TF in hospitalized COVID-19 patients compared with HC (FIG. 1).

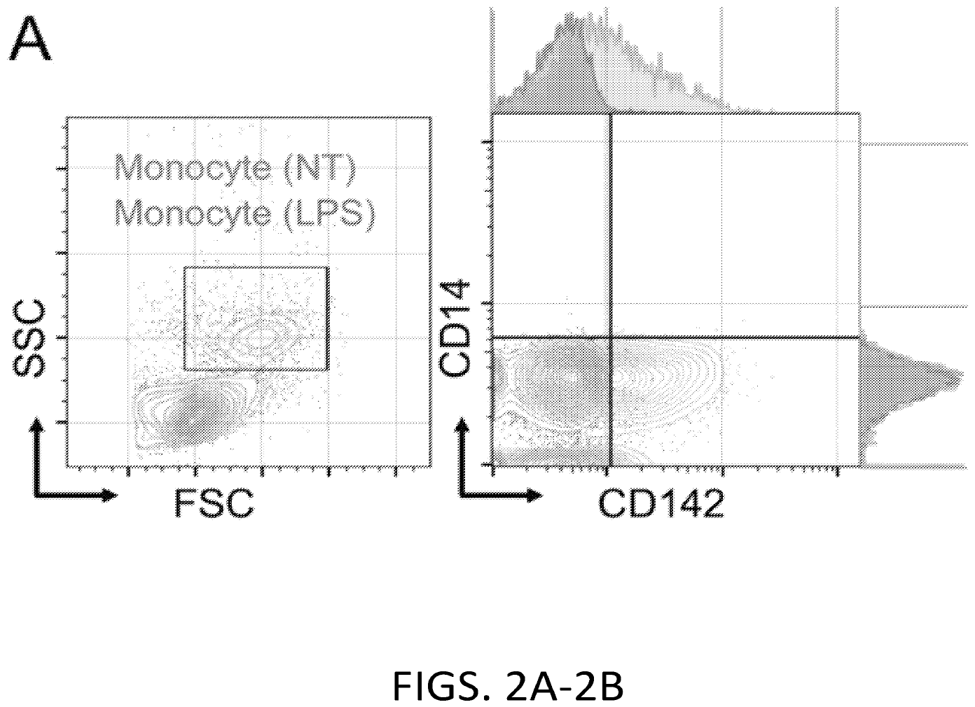

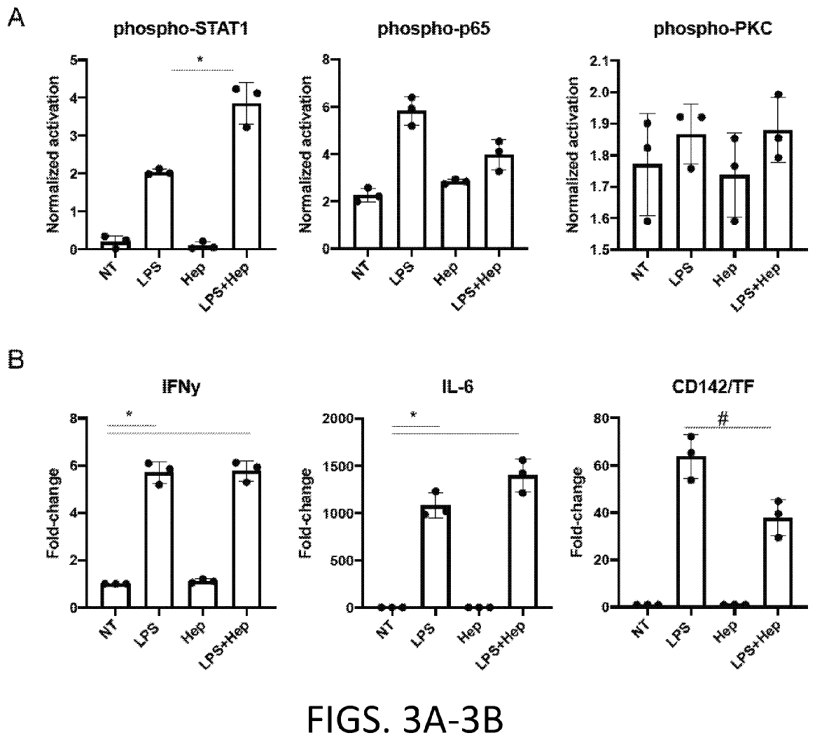

Results of the present disclosure also indicated that inflammatory mediators can induce monocyte TF expression. The observation that COVID-19 patients have increased circulating TF led to the performance of various studies to explore if innate immune cell activation could be responsible for the increases in TF. In experimental models of sepsis, several lines of evidence suggest that monocytes are the primary source of TF and interestingly, inflammatory mediators themselves can increase TF expression in circulating monocytes. To determine if inflammatory mediators can drive monocyte TF expression, CD14+ monocytes were isolated from peripheral blood mononuclear cells (PBMC) and stimulated with lipopolysaccharide (LPS; 1 μg/m1) for 2 and 4 hours. Using flow cytometry and qRT-PCR, data indicated that LPS rapidly drives TF expression (both at the protein and transcriptional level) (FIG. 2 and FIG. 3). As described herein, TF is unique in that the presence of TF alone does not indicate TF activity. To assess if monocyte-bound TF is active, an FXa activity assay was employed. CD14+ monocytes were stimulated with LPS (with and without heparin) and FXa activity was measured to investigate whether induction of TF could drive the thrombosis associated with COVID-19.

Results of the present disclosure also indicated that heparin rapidly modulates inflammatory monocyte activation. Heparin is a naturally occurring molecule used primarily because of its anti-thrombotic activity. In vitro and animal studies have suggested that heparin also has immune-modulating properties. To investigate the potential mechanisms through which heparin could modulate innate immune responses, PBMCs were isolated and treated with LPS with or without heparin. Heparin was not cytotoxic at the concentrations and timepoints evaluated (FIG. 3). Heparain alone had no effects on signaling pathway activation or the inflammatory transcriptional programs probed in PBMC (FIG. 3). However, co-treatment of PBMC with LPS and heparin led to augmentation of type I interferon signaling through increases in STAT1 activation (FIG. 3). From a transcriptional standpoint, short-term heparin treatment seemed to blunt transcriptional induction of TF by LPS (FIG. 3). Recent studies have emphasized the importance of early STAT1 signaling in containing SARS-CoV-2 infections and these data indicate that heparin could augment innate immune responses in the early stages of COVID-19.

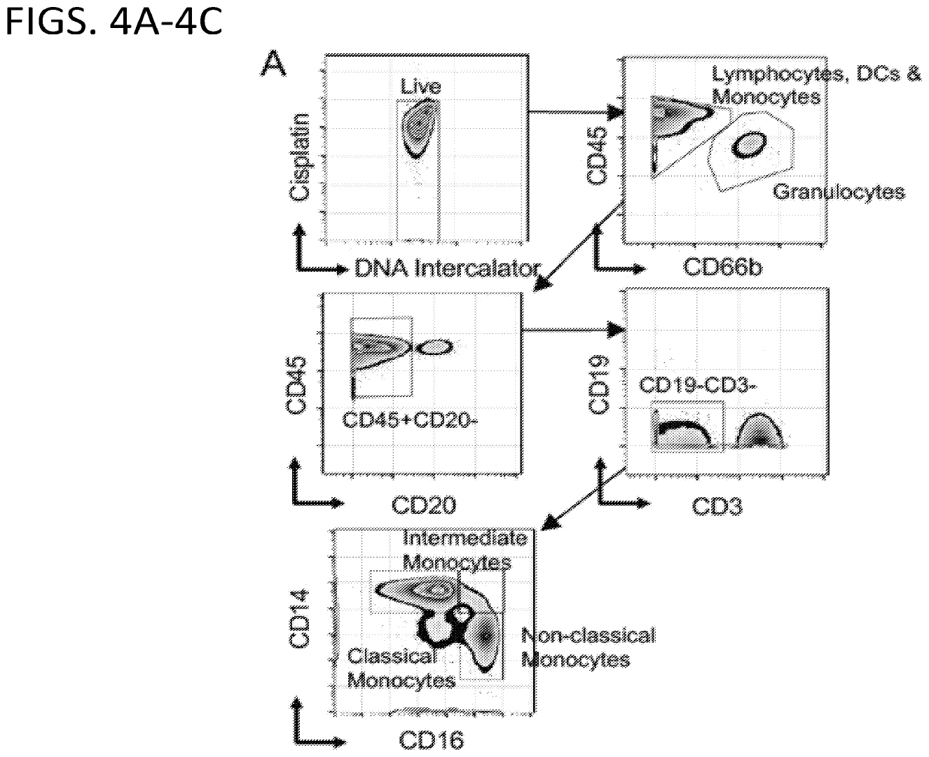

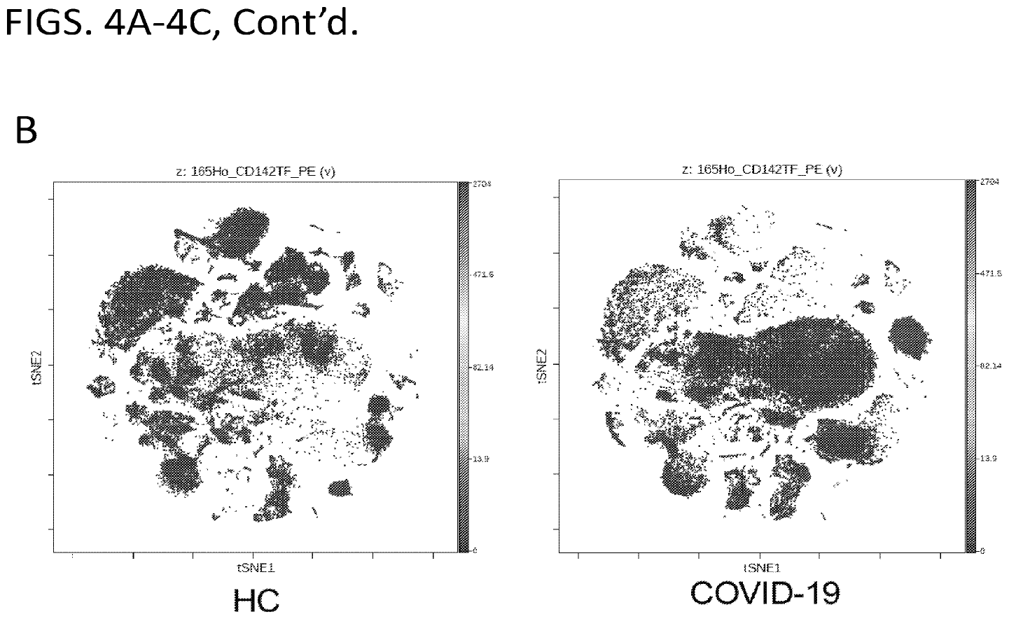

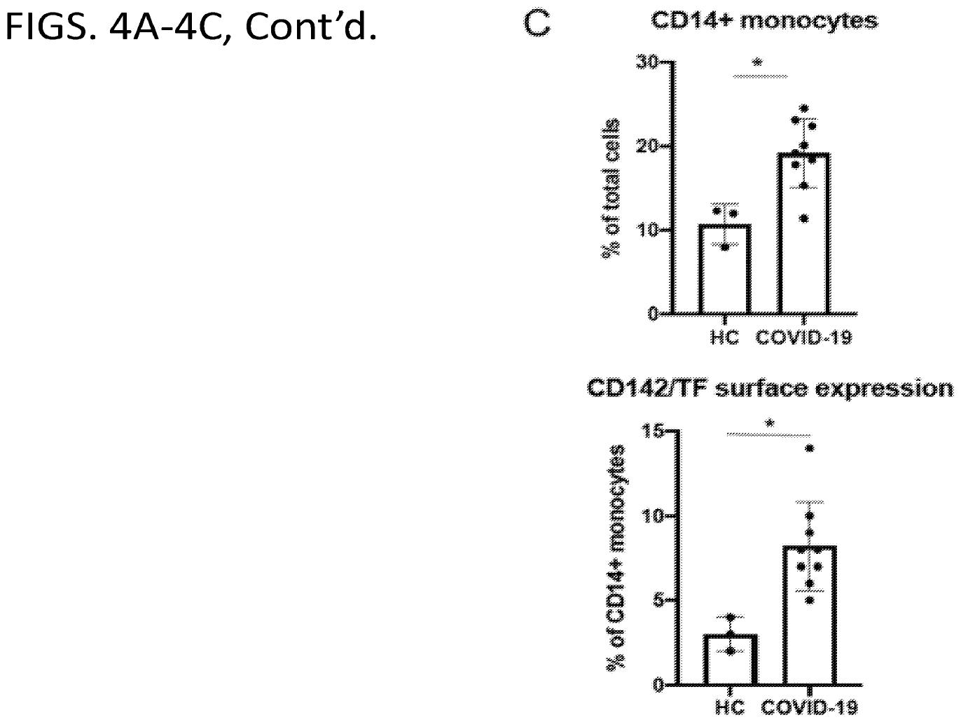

Results of the present disclosure also indicated that patients who have recovered from SARS-CoV-2 have residual increases in monocyte TF expression. Because of the striking increases in monocyte-TF upon exposure to inflammatory mediators and the increased circulating TF in hospitalized COVID-19 patients, studies were conducted to examine if patients with COVID-19 had evidence of increased TF/CD142 on circulating monocytes. Because little is known about residual immune activation in COVID-19 patients during recovery, circulating immune cell populations were profiled in patients who were previously infected with SARS-CoV-2 and compared to HC. Interestingly, patients who have recovered from COVID-19 still have increases in all monocyte populations compared with controls and they also have increased TF/CD142 expression (FIG. 4). These results suggest that even in the convalescent stage of COVID-19, residual immune dysfunction could predispose patients to immunothrombotic complications.

Innate immune responses serve as the first line of antiviral defense and are essential to develop immunity against viral pathogens. Observational clinical studies have found evidence of local and systemic inflammation and dysfunctional Mϕ (macrophage) activation in SARS-CoV-2 infections. In addition to immune dysregulation, clinical studies have found evidence of coagulation disturbances in COVID-19 patients. Small observational studies suggest that therapeutic-dose heparin may improve clinical outcomes in patients with SARS-CoV-2 infections, and these findings were the basis for the ATTACC randomized control trial (RCT) designed to directly assess the clinical benefits of therapeutic-dose heparin in COVID-19 patients.

ATTACC afforded a unique opportunity to understand the mechanisms underlying the reciprocal regulation of inflammation and thrombosis in COVID-19. One objective of the present disclosure was to define the cellular and functional phenotypes of circulating CD14+ and TF/CD142+ immune cells from SARS-CoV-2 patients, including how these phenotypes evolve during the inpatient management of SARS-CoV-2 infections, and to further understand how these immune phenotypes are modulated by parenteral anticoagulation. It was hypothesized that cytokines and ATII promote inflammatory Mϕ activation, increasing CD142+ expression by Mϕ, and that these innate immune responses are dampened by parenteral anticoagulation with heparin in COVID-19 patients. As provided further herein, understanding the mechanisms underlying immunothrombosis informed the development of novel diagnostic and therapeutic approaches for immunothrombotic diseases. The outcome measures described in the present disclosure included mass cytometry profiling of circulating monocytes and proteomic/transcriptomic profiling of innate immune programs that drive immunothrombosis in COVID-19.

The ATTACC inclusion and exclusion criteria are described in Table 1. The studies described herein help define the relationship between circulating immune cells and CD14+ monocytes in COVID-19 patients. Because of the relative paucity of information on the longitudinal changes in immune populations, the sample size for this study was estimated from a recent study that examined changes in total monocytes during SARS-CoV-2 infections (in relation to symptom onset). In a published study of 112 moderate-severity COVID-19 patients, total monocyte counts were increased early in the infection compared with HC (17% vs 9%) and in 40 severe COVID-19 patients, total monocyte counts increased early in the infection (16% vs 9%). Conservatively assuming a within-patient correlation of 0.4 between enrollment and at day 3, a sample size of 75 patients provides >80% power at alpha 0.05 to detect a similar change in monocyte frequencies by McNemar's test. Healthy control subjects were recruited through the University of Michigan; all potential controls will undergo SARS-CoV-2 nasopharyngeal swab (RT-qPCR) and serology testing to exclude asymptomatic carriers from the HC which would confound the analyses. Clinical variables and laboratory values were abstracted from the ATTACC clinical database and analyzed in parallel with immune phenotyping assays described herein. In addition to comparing COVID-19 patients to controls, the hospitalized COVID-19 patients profiled in herein were also categorized as moderate (O₂<9 L and non-ICU status) and severe (O₂ requirements>9 L and/or ICU-status).

TABLE 1
ATTACC inclusion/exclusion criteria.

| Inclusion Criteria | Exclusion Criteria |
|---|---|
| Patients ≥18 years of age | Receiving invasive mechanical ventilation |
| who require hospitalization | Active bleeding |
| anticipated to last ≥72 hours, | Risk factors for bleeding, including platelet |
| with microbiologically- | count less than 50 × 10⁹/L, INR greater |
| confirmed COVID-19, enrolled | than 2.0, or baseline aPPT greater than 50, |
| less than 72 hours of hospital | hemoglobin less than 80 g/L |
| admission or of COVID-19 | Acute or subacute bacterial endocarditis |
| confirmation | History of heparin induced thrombocytopenia (HIT) |
| | or other heparin allergy including hypersensitivity |
| | Current use of dual antiplatelet therapy |
| | Patients with an independent indication for |
| | therapeutic anticoagulation |
| | Patients in whom imminent demise is anticipated and |
| | there is no commitment to active ongoing intervention |
| | Pregnancy |
| | Anticipated transfer to another hospital that is |
| | not a study site within 72 hours |
| | Enrollment in other trial related to anticoagulation |
| | or antiplatelet therapy |

Results of the present disclosure also include longitudinal profiling of inflammatory biomarkers and blood monocytes in COVID-19 patients. Basic and observation studies of SARS-CoV-2 infections have implicated both hypo- and hyperactive immune responses in the pathobiology of COVID-19. One example of this concept is with regards to Type I IFN signaling in COVID-19. Several studies have found that IFN is protective early in the disease but later becomes pathological. These studies highlight the dynamic and coordinated elements of the immune response and the importance of temporal regulation in balancing the benefits and harm inherent in immune responses. Because of temporal dynamics of immune trajectories, longitudinal profiling of the immune response is critical in understanding the immune mechanisms that underlie COVID-19 and natural history of COVID-19. ATTACC afforded the opportunity to longitudinally profile innate immune cells and relate them to clinical variables. Although it is known that activated monocytes are one of the primary sources of circulating TF, the prevalence and functional properties of CD14+/CD142+ monocytes and how monocyte subsets evolve during the course of a SARS-CoV-2 infection remains unknown. Therefore, as described further herein, one focus of the present disclosure was to use mass cytometry to define the frequencies of monocyte subsets and CD142/TF+ monocytes in COVID-19 patients. Additionally, CD142+ monocytes were correlated to traditional monocyte subsets (classical; intermediate; non-classical) and experiments were conducted to directly assess how therapeutic-dose heparin affects the frequencies of monocyte populations. Patient blood samples (approximately 10 mL) were collected at the time of randomization and 3 days afterwards (coinciding with the clinical assessments built in to the ATTACC trial) using plasma (for biomarker assessment), whole blood with Cytodelics stabilizer (for mass cytometry profiling), and CPT tubes (for functional profiling). The longitudinal approach employed herein enabled several parallel comparisons including: 1) the relative abundance of monocyte populations in COVID+ patients (on admission) compared with controls; 2) how the relative abundance of circulating monocyte populations evolve during hospitalization (longitudinal assessment); and 3) how does therapeutic heparin modulate relative abundance of circulating monocyte populations.

To complement the immune profiling described above, the Bio-Plex Pro™ Human Cytokine Screening Panel was used to interrogate 48 inflammatory mediators in tandem with free TF and ATII. Quantifying cytokines in tandem with circulating immune cells allow for the categorization of immune trajectories to type 1/2/3 immune responses. Profiling immune cell populations with mass cytometry provides both breadth and depth; and the optimized human immune panel allows for parallel assessment of markers of cell identity and function (lineage, adhesion, migration, and both cell-surface/intracellular activation) (Table 2). To minimize technical variability (processing, staining, and instrument), Pd CD45 barcoding was, which allows for the processing of pooled and batched samples. For the mass cytometry profiling experiments, patient samples were collected and stored in tubes with stabilizing agent (Cytodelics; 2 mL). The antibody staining protocols for mass cytometry mirror that of flow cytometry. CyTOF1 mass cytometer (DVS/Fluidigm) was used for the data acquisition. PBMCs were assessed after the exclusion of dead cells and doublets. The three main monocyte subsets of classical (˜85%), intermediate (˜5%), and non-classical (˜10%) monocytes were characterized by the level of CD14, CD16, and HLA-DR (classical: CD14^{High}CD16^{Low}; intermediate: CD14^{High}CD16^{High}; non-classical CD14^{Low}CD16^{High}).

TABLE 2
Mass cytometry human markers.

| Human Antibody | Function |
|---|---|
| CD1c | Cell-lineage |
| CD2 | Cell-lineage |
| CD3 | Cell-lineage |
| CD4 | Cell-lineage |
| CD7 | Cell-lineage |
| CD8 | Cell-lineage |
| CD9 | Adhesion |
| CD11b | Adhesion |
| CD11c | Adhesion |
| CD14 | Adhesion |
| CD16 | Adhesion |
| CD19 | Cell-lineage |
| CD36 | Scavenger Receptor |
| CD142/TF | Tissue Factor |
| CD40 | Activation |
| CD44 | Adhesion |
| CD45 | Cell-lineage |
| CD63 | Activation |
| CD64 | Activation |
| CD74 | Antigen Presentation |
| CD80 | Activation |
| CD86 | Activation |
| CD123 | Cell-lineage |
| CD161 | Cell-lineage |
| CD169 | Adhesion |
| HLA-DR | Antigen Presentation |
| CCR2 | Migration |
| CX3CR1 | Migration |
| p-p38 | MPAK |
| p-STAT1 | Jak-Stat |
| p-STAT3 | Jak-Stat |
| p-STAT5 | Jak-Stat |
| p-p65 | NFκB |
| Btk/Itk | INFα induced |

Results of the present disclosure also includes functional profiling of CD14+ monocyte responses in COVID-19 patients. Targeted proteomics and transcriptomics were used to define the functional responses of CD14+ and CD14+/CD142+ monocytes, and how the monocyte functional responses are modulated by both SARS-CoV-2 infection and therapeutic-dose heparin. A significant proportion of individual variation in immune responses is only identifiable after a stress or stimuli, reinforcing the notion that the immune system is inherently context specific. To explore these functional differences and to complement the biomarker/immune cell identity profiling described above, PBMCs were sorted using both CD14 and CD142 and stimulated with LPS (TLR4), ssRNA40 (TLR7/8), ATII for 4 hours and the stimulated monocytes were compared with untreated controls. Both TLR4 ligands and TLR7/8 were used to capture stereotyped innate immune responses to bacterial (TLR4) and viral pathogens (TLR7/8). About 10 million cells per CPT tube were obtained, of which approximately 10% are CD14+ monocytes. The microfluidic proteomic platform and RNA seq transcriptomics used are well suited for assessing this number of cells. After stimulation, RNA and protein were isolated and processed for transcriptomics (QuantSeq; Lexogen) and proteomics (microfluidic immunoblotting). QuantSeq uses Illumina Read 1 linker sequence in the second strand synthesis primer, generating next generation sequence reads towards the poly(A) tail, directly reflecting the mRNA sequence. QuantSeq NGS is well suited for differential gene expression workflows such as those described herein. With respect to the specific signaling pathways, the microfluidic proteomic platform described herein was developed, and it was used to probe for activation of innate immune signaling pathways (MAPK, SP1, NFkB, Jak-STAT, IRF3, Akt, mTor, and PKA). The nature of the sorting protocol allows for the comparison of functional responses (transcriptomic and signaling pathway activation) between CD14+ and CD14+/CD142+ monocytes and how these functional responses are modulated by both SARS-CoV-2 infection and therapeutic-dose heparin. It was hypothesized that inflammatory mediators and ATII promote Mϕ (macrophage) inflammatory and thrombotic programs and that parenteral anticoagulation with heparin dampens both of these innate immune responses. More specifically, these data demonstrate that heparin directly augments STAT1 signaling in monocytes and this augmentation helps mount a more robust type I IFN response, which has been hypothesized to be critical in controlling early SARSCoV-2 infections.

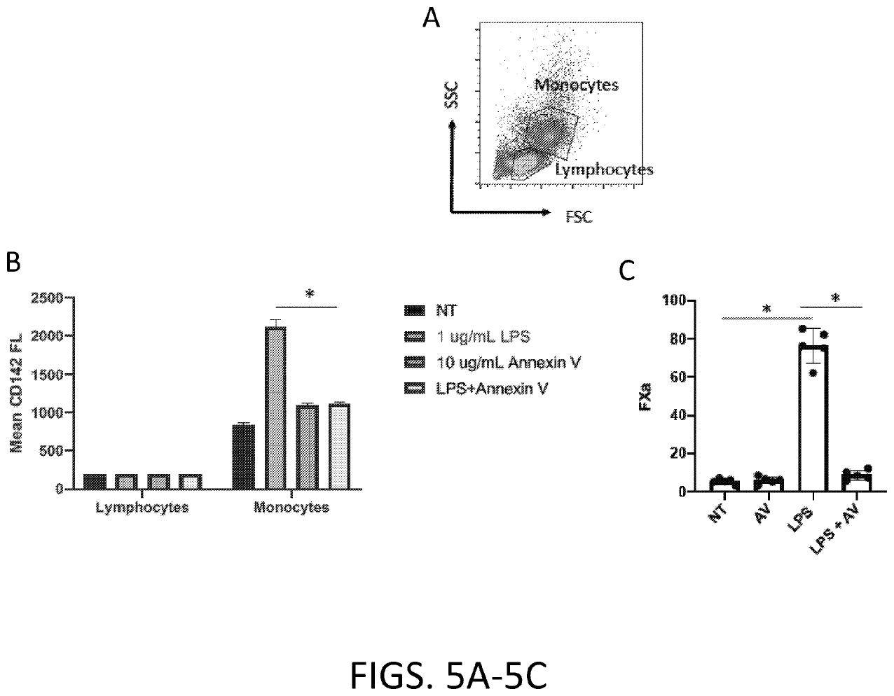

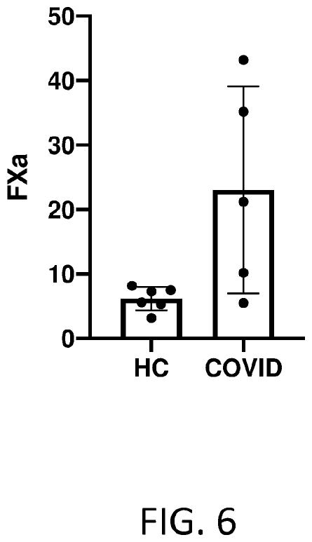

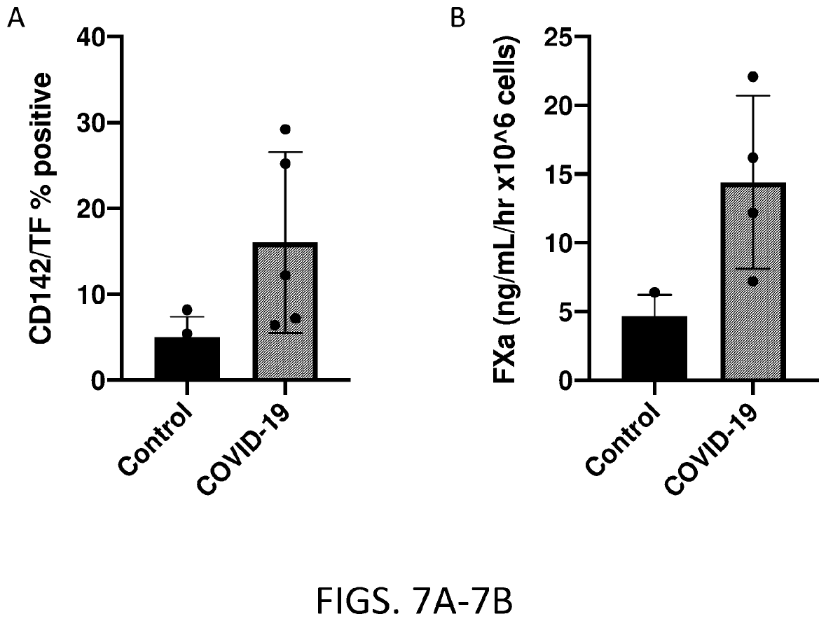

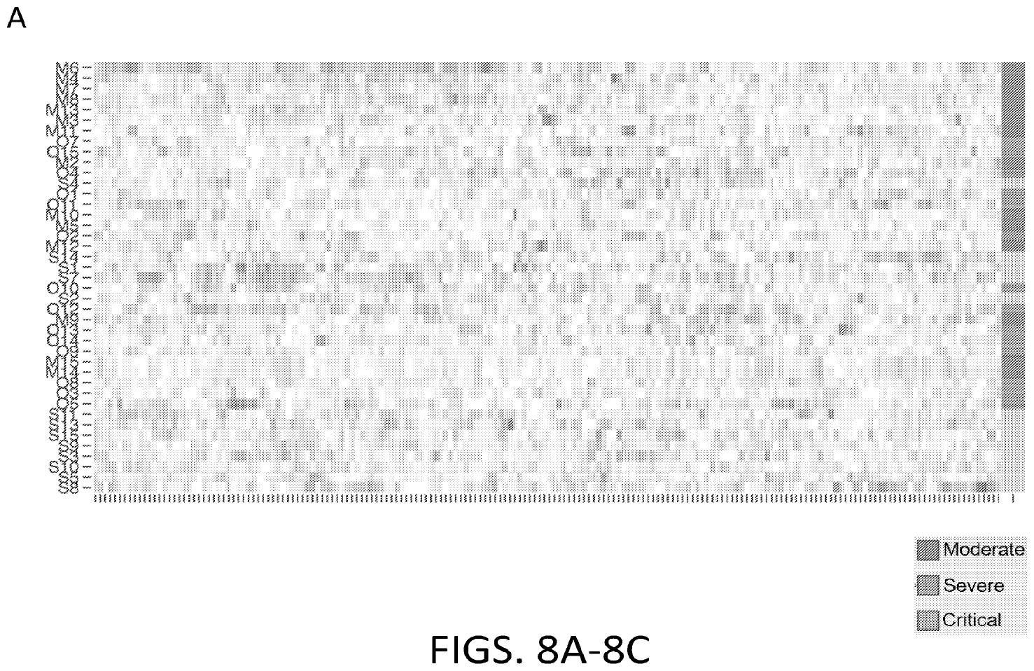

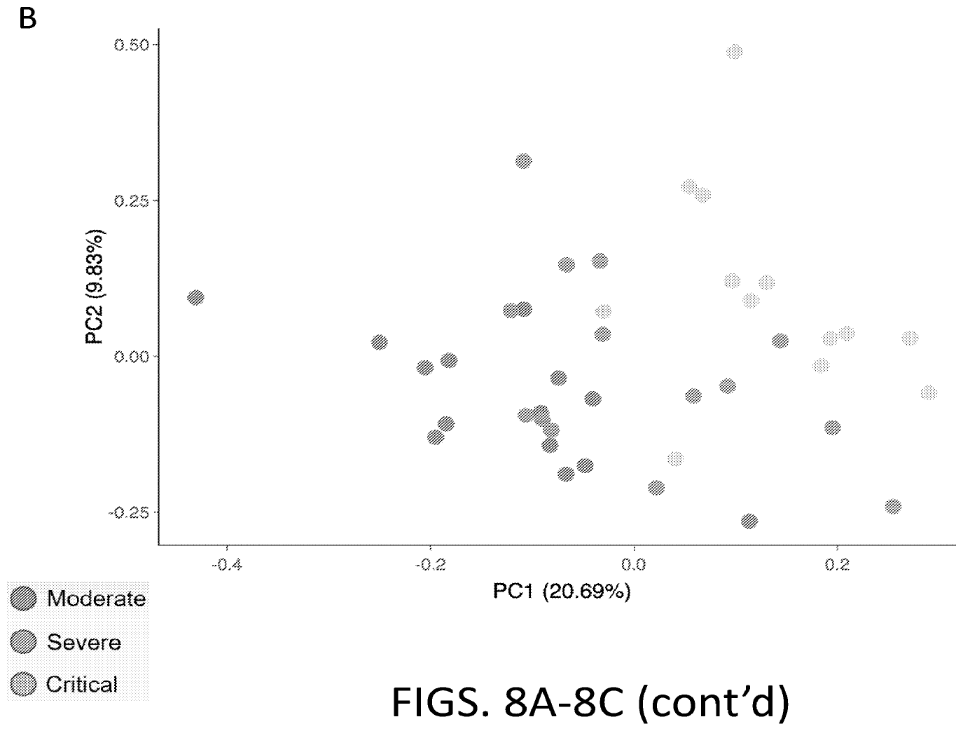

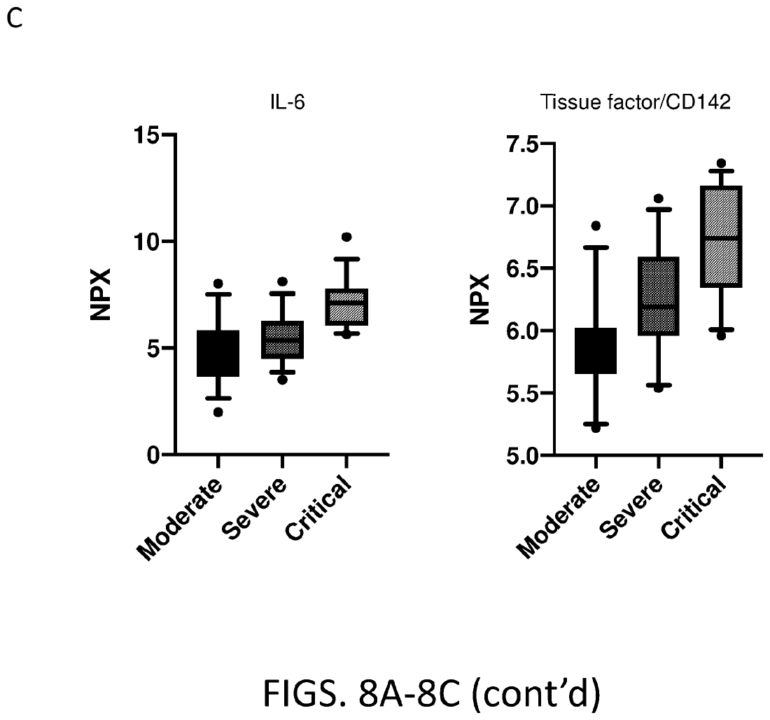

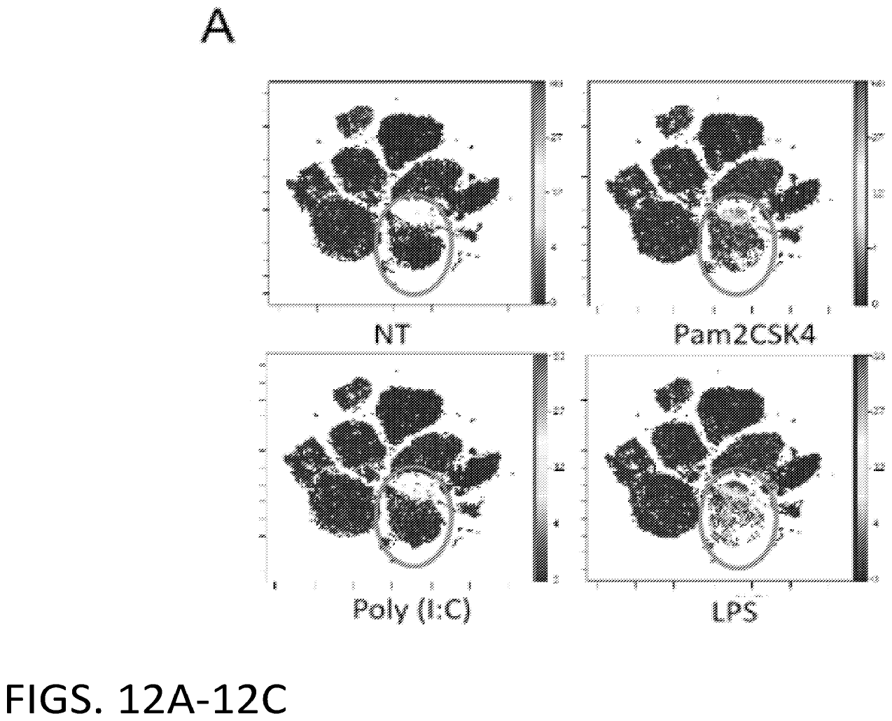

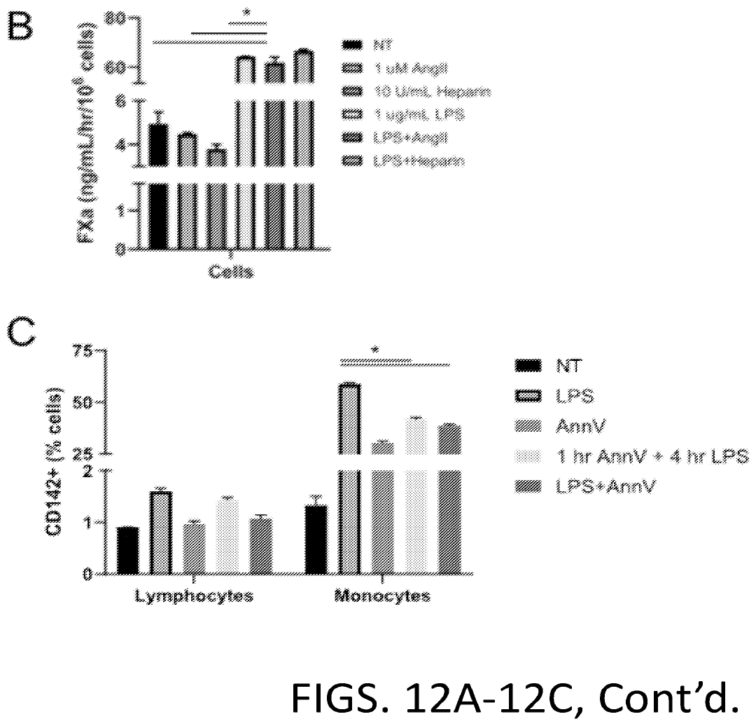

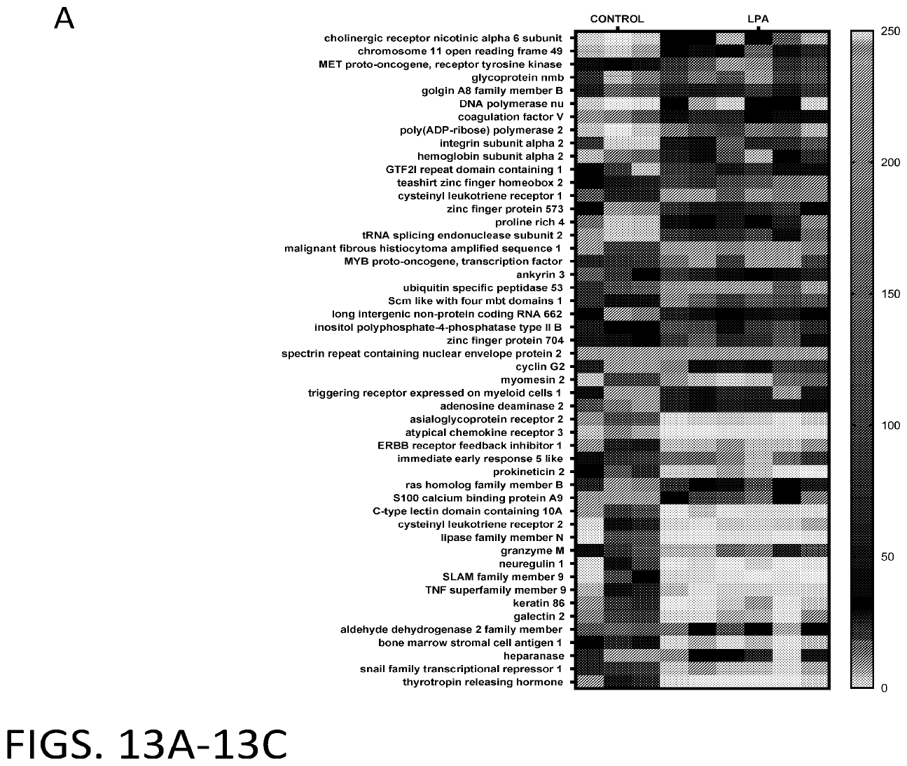

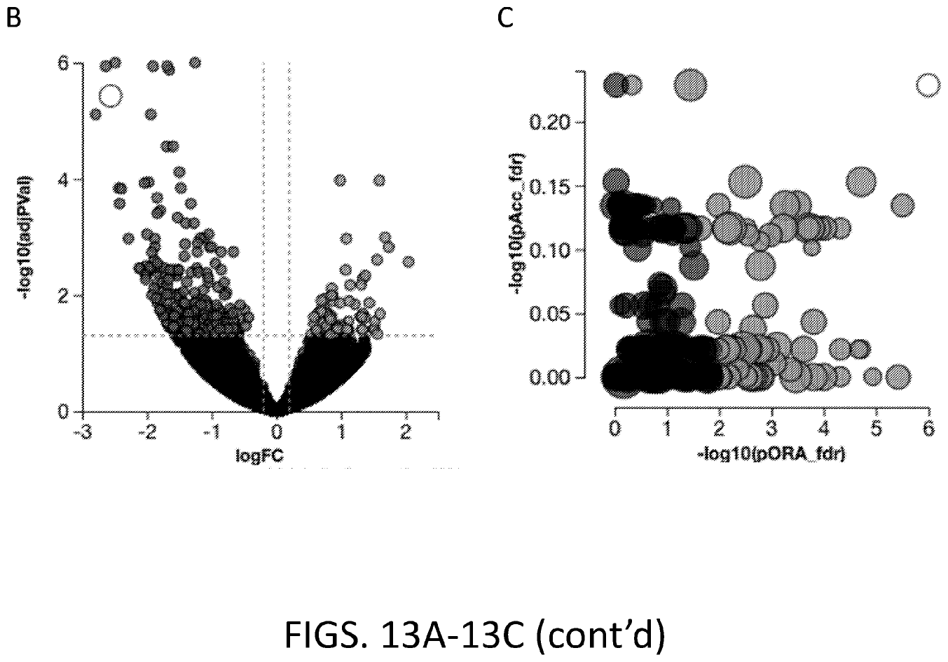

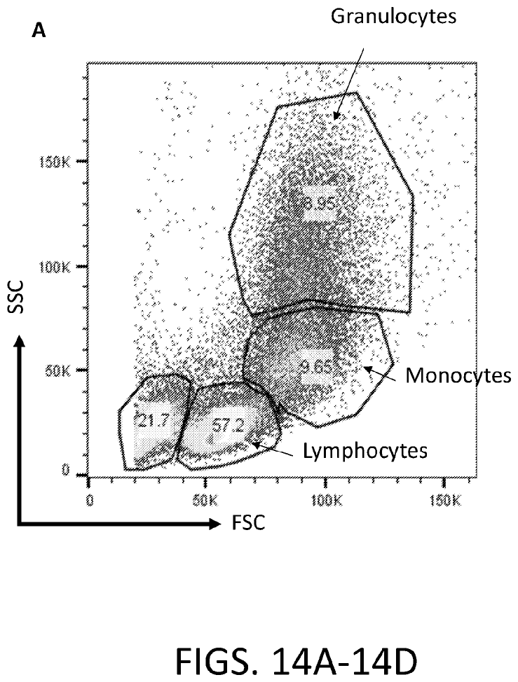

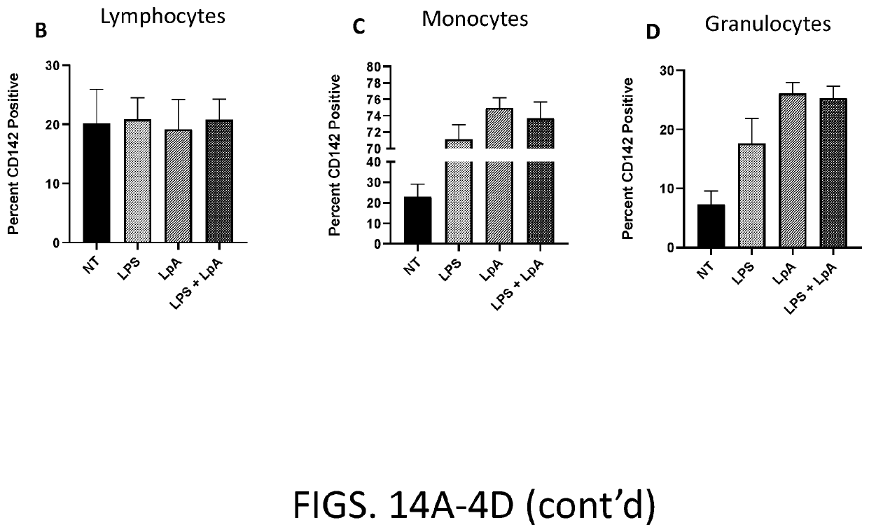

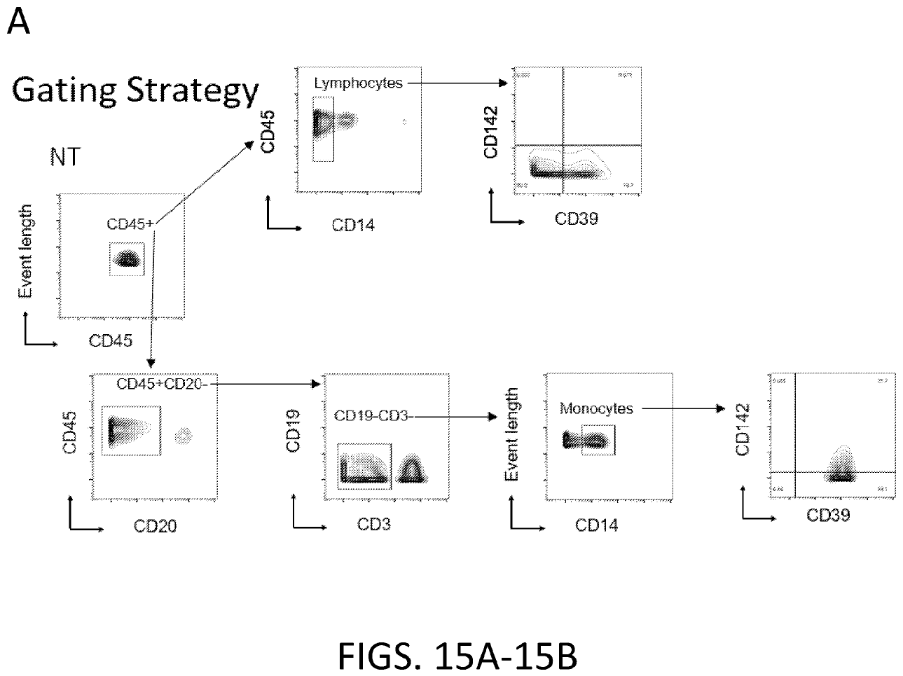

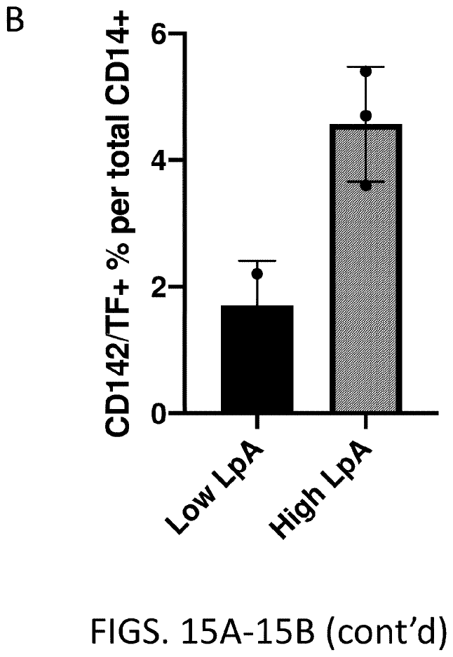

To assess if Mϕ-bound TF is active, an FXa activity assay was employed. PBMCs were stimulated with LPS and FXa production was quantified to assess TF activity. Results indicated that not only does LPS induce TF expression in Mϕ, but it also increases TF activity. Because PBMC are heterogeneous and include lymphocytes, platelets, and other circulating particles, CD14+ Mϕ were purified and probed for TF induction in response to LPS; surprisingly, results indicated much less TF induction compared with bulk PBMC stimulation, suggesting that cell-cell or cell-particle interactions were critical in driving Mϕ TF induction. A recent study demonstrated that COVID-19 is associated with increases in circulating apoptotic cells and platelet microparticles (which bear the apoptotic marker PS); therefore, it was hypothesized that detection of both circulating TLR ligands and apoptotic cells may be important in driving TF expression. To test this, PBMCs were pretreated with annexin V to block PS detection prior to LPS stimulation. Results indicated that that masking Mϕ apoptosis sensing resulted in a dramatic blunting of TLR-driven TF expression (see FIG. 5 and FIG. 12). These findings corroborated clinical observations that TF can be induced by inflammatory mediators and that Mϕ themselves rapidly induce TF expression in response to TLR ligands and also support the hypothesis that Mϕ TF could underlie the thrombosis associated with COVID-19.

To assess residual thrombotic risk in convalescent COVID-19 patients, 8 COVID-19 patients were profiled using mass cytometry. Results indicated that even in recovered COVID-19 patients, there was increased CD14+ Mϕ and increased TF+ Mϕ compared with HC, reinforcing that thrombotic risk persists even during recovery.

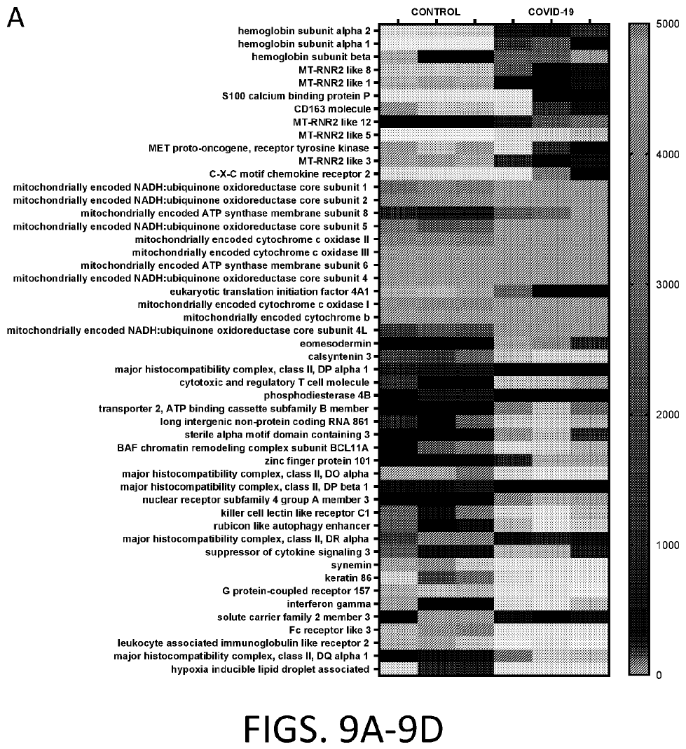

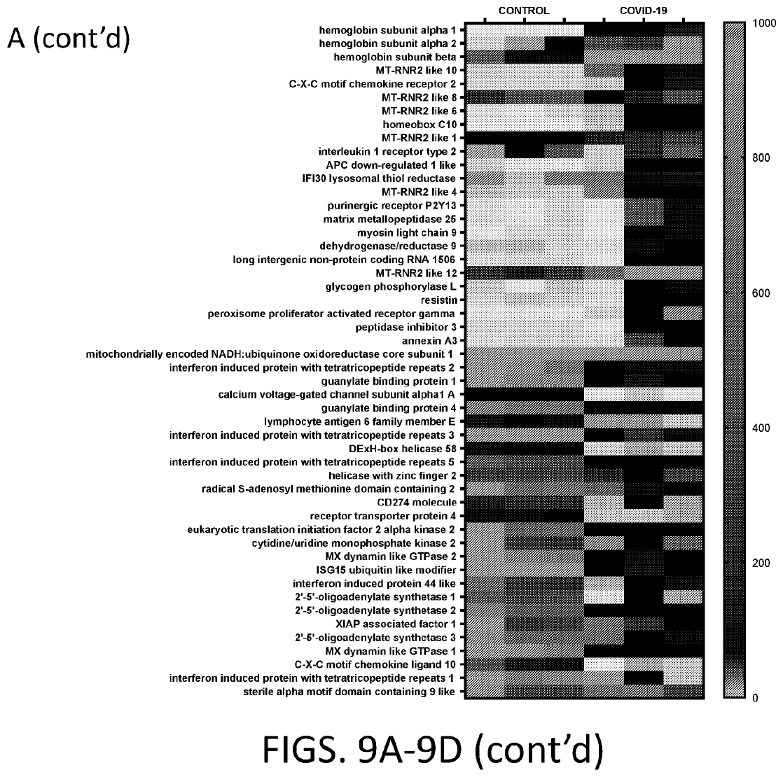

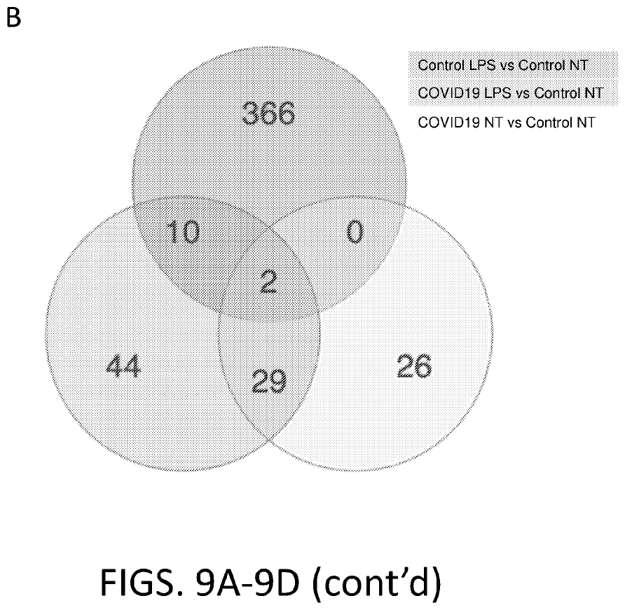

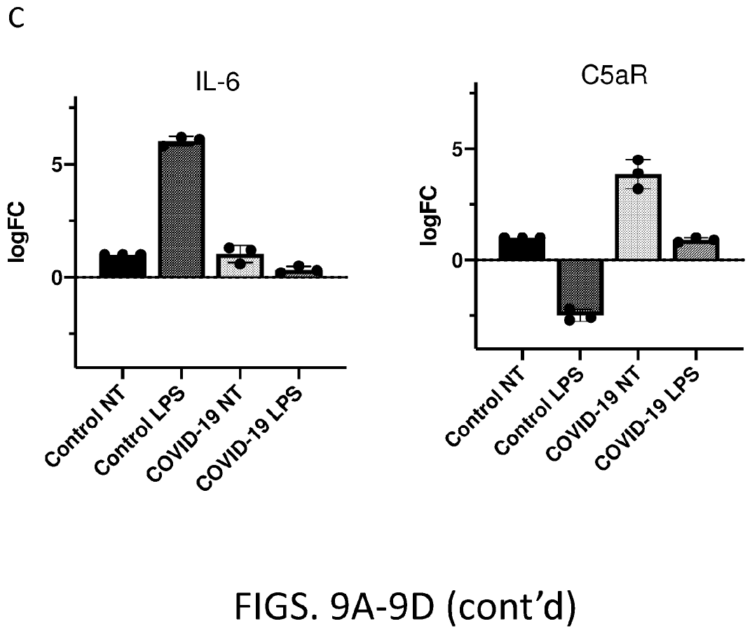

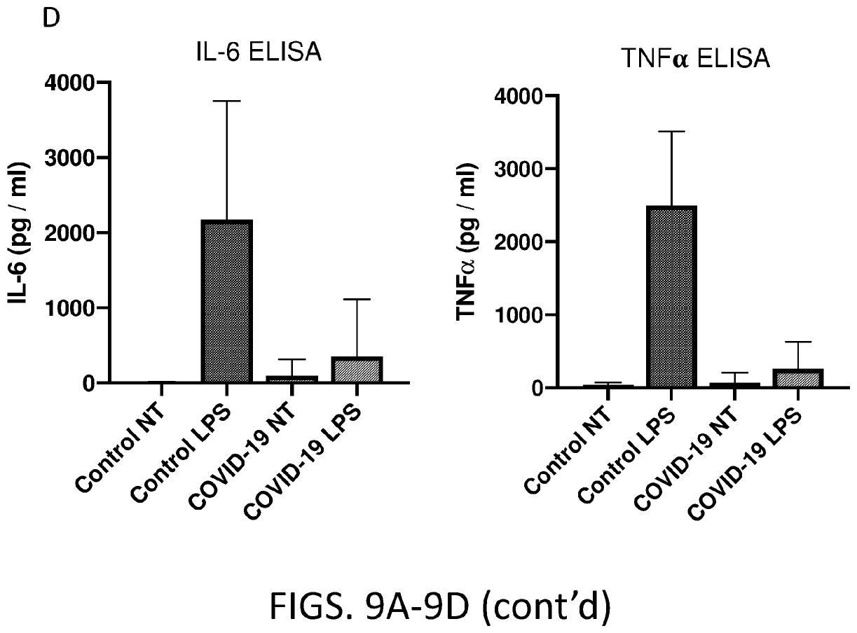

Results of the present disclosure also indicated that COVID-19 is associated with elevated inflammatory cytokines. Numerous studies have found that patients hospitalized with COVID-19 have elevated inflammatory markers. To characterize the patient cohort and to put it into context with other observational studies, Luminex assays were run on plasma from the COVID-19 cohort. In agreement with other studies, results demonstrated that IL-6 and TNFα were elevated in the COVID-19 cohort compared to the control subjects [median (IQR), all units pg/ML; IL-6: 4.65 (3.32-9.16) vs 0.69 (0.55-0.89), p<0.001; TNFα: 4.49 (1.87-8.03) vs 0.04 (0.04-0.84), p<0.001] (FIG. 9). IL-6 and TNFα are classical inflammatory markers and their upregulation is indicative of a robust immune response; however, sustained elevations of inflammatory mediators suggest a hyperactive immune response, which ultimately can be detrimental to the host.

Results of the present disclosure also indicated that COVID-19 is associated with elevated GAC degradation products. Hyperactivation of the immune system is a common manifestation of SARS-CoV-2 infections. In parallel, SARS-CoV-2 can cause epithelial and endothelial damage. In addition to elevation in inflammatory cytokines, results indicated that GAC components, SDC1 and Hyal, were elevated on admission compared to controls [median (IQR), all units pg/ML; SDC1: 247.37 (101.43-458.26) vs 84.8 (52.88-123.59), p=0.036; Hyal: 26.41 (16.4-35.1) vs 3.01 (1.66-4.61) p<0.001]. Correlations between markers of immune activation and the GAC biomarkers SDC1 and Hyal in the COVID-19 cohort were also found. Importantly, although there were significant associations between markers of inflammation and GAC disruption (IL-6 and IL-8 with both Hyal and SDC1), there was also heterogeneity (e.g., TNFα with Hyal but not SDC1) suggesting specific interactions between different arms of the immune response and GAC degradation. These results indicate that inflammatory biomarkers (e.g., IL-6 and TNFoc) and GAC markers (e.g., Hyal and SDC1) were elevated in hospitalized COVID-19 patients compared with controls. Additionally, it was found that not all circulating immune markers are associated with GAC remodeling, further reinforcing the notion that vascular and immune axes are regulated specifically and are dysfunctional in severe SARS-CoV-2 infections.

Although a majority of COVID-19 patients are either asymptomatic or have only mild symptoms, a subset of COVID-19 patients develops severe respiratory symptoms and ARDS. The reason for the heterogeneous clinical presentation and the pathologic mechanisms that underlie respiratory failure still are not fully understood. Recent studies have suggested several mechanisms linked to vascular function that may contribute to the progression of COVID-19. First, to enter cells, SARS-CoV-2 binds to the ACE2 receptor which impairs ACE2 activity. Impaired ACE2 activity leads to elevated and sustained angiotensin vascular dysfunction and also indirectly activates the kallikrein—bradykinin pathway which leads to in increased vascular permeability. Second, activated immune cells are recruited to pulmonary vasculature and produce reactive oxygen species (ROS), inflammatory cytokines, and other mediators which can disrupt the pulmonary vascular barrier. Finally, SARS-CoV-2 can have direct and indirect effects on pulmonary endothelial cells themselves. SARS-CoV-2 can trigger endothelial inflammation and the release of inflammatory cytokines production such as IL-1β, TNFα, and IL-6. These inflammatory cytokines can activate enzymes that degrade the endothelial GAC, resulting in endothelial dysfunction, a prothrombotic endothelial phenotype, and cell death, all of which are hallmarks of ARDS and present in severe COVID-19.

Taken together, circulating GAC degradation products were elevated in hospitalized COVID-19 patients suggesting that vascular injury and GAC remodeling contributes to the morbidity and mortality in SARS-CoV-2 infections. The parallel assessment of GAC markers with inflammatory cytokines may help to identify patients at risk of COVID-19 complications and further understanding of the bidirectional relationship between specific immune and GAC responses in COVID-19 are certain to advance understanding of how inflammation and vascular dysfunction coexist. However, these findings further support the notion that vascular dysfunction occurs in tandem with immune dysregulation and could be leveraged to identify patients who are at high risk and could identify novel therapeutic approaches towards COVID-19 and other immune-vascular diseases.

### 3. Lipoprotein(a)

Elevated circulating levels of lipoprotein (a) (Lp(a)) accelerate atherogenesis and increases the risk for atherothrombotic events. Lp(a) resembles low density lipoprotein (LDL) and consists of a lipoprotein moiety and the plasminogen-related glycoprotein, apo(a). However, the surprisingly large impact of Lp(a) compared with LDL on atherosclerotic cardiovascular disease (ASCVD) risk suggest that Lp(a) possesses other pathogenic properties. The pathogenicity of Lp(a) is thought to reflect its ability to accelerate inflammation, and potentiate thrombosis through impaired fibrinolysis.

Evidence from experimental models and clinical studies has demonstrated that Lp(a) is the primary lipoprotein carrier of oxidize phospholipids (OxPL). OxPL is a danger associated molecular pattern (DAMP), which can be recognized by pattern recognition receptors (PRRs) on innate immune cells. Activation of these cascades trigger inflammation, thrombosis, and plaque destabilization. In observational studies and clinical trials of high-intensity statin therapy, the level of OxPL on apoB-containing lipoproteins (primarily Lp(a)) was found to be predictive of future ASCVD risk after adjustment for Lp(a) concentration and major coronary heart disease (CHD) risk factors. A secondary analysis of the Assessment of Clinical Effects of Cholesteryl Ester Transfer Protein Inhibition With Evacetrapib in Patients at a High Risk for Vascular Outcomes (ACCELERATE) trial demonstrated that Lp(a) levels are most predictive of ASCVD events in patients with systemic inflammation (hs-CRP>2 mg/mL), reinforcing the mechanistic link between Lp(a) and immune dysregulation. An integrated conceptual understanding of how Lp(a) orchestrates thrombosis, inflammation, and atherogenesis with clinical variables and phenotypes, will allow for more nuanced, causally linked assessments of ASCVD residual risk.

The recognition that macrophages (Mϕ), a key cellular orchestrator of inflammation, can also mediate thrombosis, provides a novel lens through which to understand the physiological and pathological dimensions that regulate both coagulation and inflammation. Embodiments of the present disclosure employ systems immunology approaches (targeted microfluidic proteomic platform, mass cytometry, RNA-seq) to profile Mϕ phenotypes in subjects with coronary heart disease (CHD). As described further herein, the relationship between Lp(a) and circulating immune cells, monocyte subsets and circulating thrombotic and inflammatory mediators was analyzed in subjects with clinically stable coronary heart disease (CHD).

One objective of the present disclosure was to understand how Lp(a) regulates monocyte phenotypes, triggers monocyte-mediated inflammation, and mediates immune-mediated thrombosis. Targeted proteomics and transcriptomics were used to define the mechanisms through which Lp(a) and inflammatory mediators (LPS and TLR4, ssRNA40 and TLR7/8) drive Mϕ TF expression and activity. Second, the relative distribution of TF in plasma, microvesicles, and circulating monocytes was analyzed in CHD patients with elevated Lp(a) levels. Third, the mechanisms through which Lp(a) activates monocytes and induces TF expression/activity were defined. Overall, these studies are the first to link monocyte activation to both thrombosis and inflammation in persons with high Lp(a) concentrations, and thus demonstrate a novel mechanism contributing to Lp(a) associated ASCVD risk.

Results. As described further herein, embodiments of the present disclosure demonstrate that Lp(a) is a predominantly genetically-determined yet modifiable heterogeneous lipoprotein with multifarious properties at the nexus of inflammation and thrombosis. LPA promotor activity is upregulated by IL-6, which triggers clonal proliferation of inflammatory monocytes and macrophages. Lp(a) increases inflammatory gene expression via a TLR-mediated pathway. Lp(a) mediated inflammation triggers tissue factor expression on mononuclear cells via a TLR2-dependent pathway. And, lowering Lp(a) via IL-6 receptor blockade or treatment with PCSK9i, mRNA inhibitors reduces monocyte driven inflammation.

The study population included x CHD patients with Lp(a) concentrations >150 nmol/L (cases) and y controls (<75 nmol/L) from an outpatient cardiology practice, as shown in Table 3 below.

TABLE 3
Demographic and clinical characteristics of the patients
in high Lp(a) and control groups for pilot Lp(a) study.

| | | |
|---|---|---|
| | High Lp(a) Group | Control Group |
| Characteristics | (N = 14) | (N = 5) |

| | | |
|---|---|---|
| Age-yr | 60.6 ± 12.2 | 65.4 ± 10.3 |

| | | | | |
|---|---|---|---|---|
| Female sex-No. (%) | 6 | (42.9) | 3 | (60.0) |
| Race-No. (%) | | | | |
| White | 9 | (64.3) | 3 | (60.0) |
| Black | 2 | (14.3) | 1 | (20.0) |
| Asian | 1 | (7.1) | 1 | (20.0) |

| | | |
|---|---|---|
| Lp(a) level (nmol/L) | 289.04 ± 135.67 | 21.92 ± 15.80 |

| | | | | |
|---|---|---|---|---|
| Smoking-No. (%) | | | | |
| Current | 1 | (7.1) | 0 | (0) |
| Former | 6 | (42.9) | 1 | (20.0) |
| Never smoker | 7 | (50.0) | 4 | (80.0) |
| Diabetes-No. (%) | 2 | (14.3) | 1 | (20.0) |
| Pre-diabetes-No. (%) | 3 | (21.4) | 1 | (20.0) |
| Hypertension-No.(%) | 8 | (57.1) | 2 | (40.0) |
| CAD-No. (%) | 14 | (100) | 5 | (100) |
| Carotid disease-No. (%) | 5 | (35.7) | 1 | (20.0) |
| PAD-No.(%) | 3 | (21.4) | 0 | (0) |
| Family Hx-No. (%) | | | | |
| Premature CAD | 8 | (57.1) | 0 | (0) |
| Not premature CAD | 5 | (35.7) | 2 | (40.0) |

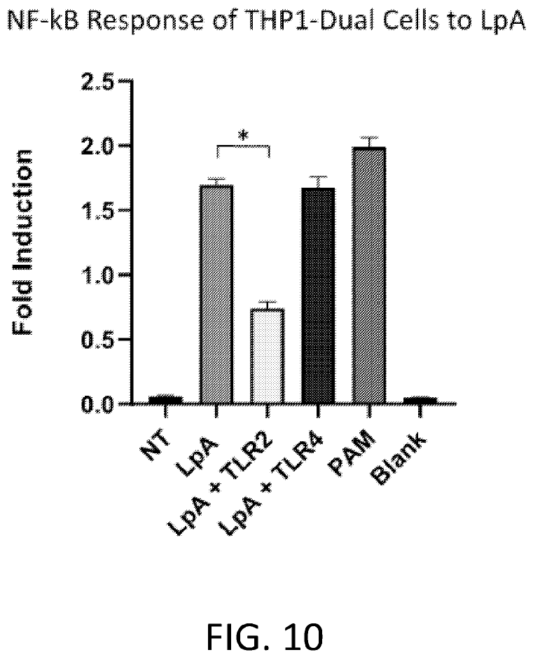

Results of the present disclosure indicate that proteomic profiles show increased immune and vascular markers. CHD patients have elevated vascular and inflammatory markers and studies suggest that Lp(a) itself can amplify vascular dysfunction and inflammation. To define the plasma protein signature of CHD patients with high vs. low Lp(a) concentrations, Olink proteomics was used. Out of the 184 protein markers probed, results indicated striking differences in CHD subjects with an elevated Lp(a) compared with age-matched controls (FIG. 10). Hierarchical cluster and principal component analysis (PCA) allowed for discrimination of the two cohorts of subjects. With respect to individual protein markers, subjects with an elevated Lp(a), had increased immune (chemokines/cytokines including IL-8 and TNFα) and vascular markers (PSGL1, OSM, uPA).

Experiments were also conducted to profile circulating peripheral blood mononuclear cells. Subjects with an elevated vs. low Lp(a) had increased classical monocytes (15% vs 8%; p<0.05) (FIG. 1). Targeted proteomics and transcriptomics was used to define the functional responses of CD14+ and CD14+/CD142+ monocytes, and how monocyte functional responses are modulated by Lp(a). The number of cells was assessed by RNA seq transcriptomics. To explore these functional differences and to complement the biomarker/immune cell identity profiling, PBMCs were sorted using both CD14 and CD142, and stimulated with LPS (TLR4) and ssRNA40 (TLR7/8) for 4 hours. The stimulated monocytes were compared with untreated controls using both TLR4 ligands and TLR7/8 to capture stereotyped innate immune responses to oxidized phospholipids and other endogenous DAMPs known to be elevated in CHD.

A microfluidic proteomic platform was developed and used to probe for activation of innate immune signaling pathways (MAPK, SP1, NFkB, Jak-STAT, IRF3, Akt, mTor, and PKA). The nature of the sorting protocol allowed for the comparison of functional responses (transcriptomic and signaling pathway activation) between CD14+ and CD14+/CD142+ monocytes. After stimulation, RNA and protein was isolated and processed for transcriptomics (QuantSeq; Lexogen) and proteomics (microfluidic immunoblotting). QuantSeq uses Illumina Read 1 linker sequence in the second strand synthesis primer, generating next generation sequence reads towards the poly(A) tail, directly reflecting the mRNA sequence.

Results indicated that Lp(a) increased NF-kB induction on THP1-Dual monocytes, and the addition of TLR2 decreased the activation effect of Lp(a). THP1-Dual monocytes stimulated for 24 h with 77.5 ug/mL Lp(a), 77.5 ug/mL Lp(a)+10 ug/ml TLR2, 77.5 ug/ml Lp(a)+10 ug/ml TLR4, and 100 ng/mL Pam2CSK4. After 24 h stimulation, supernatant was collected and NFkB activation was assessed by measuring the levels of SEAP using QUANTI-Blue assay. Levels of SEAP were determined by reading the optical density at 640 nm. *p-value<0.0001 (ANOVA). No significant differences between Lp(a) treated groups and TLR2/TLR4 treated groups. THP1-Dual monocytes stimulated for 24 h with 77.5 ug/mL Lp(a), 77.5 ug/mL Lp(a)+10 ug/ml TLR2, 77.5 ug/ml LpA+10 ug/ml TLR4, and 100 ng/mL Pam2CSK4. After 24 h stimulation, supernatant was collected and IRF activation was assessed by measuring the levels of Lucia luciferase using QUANTI-Luc assay. Levels of Lucia luciferase were determined by measuring the relative light units (RLUs) in a luminometer at a 100 ms reading time.

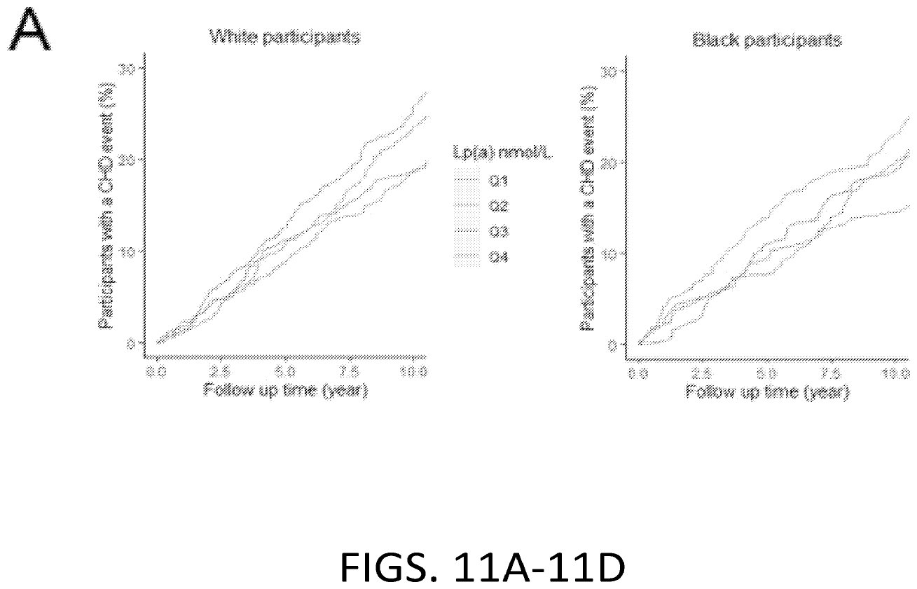

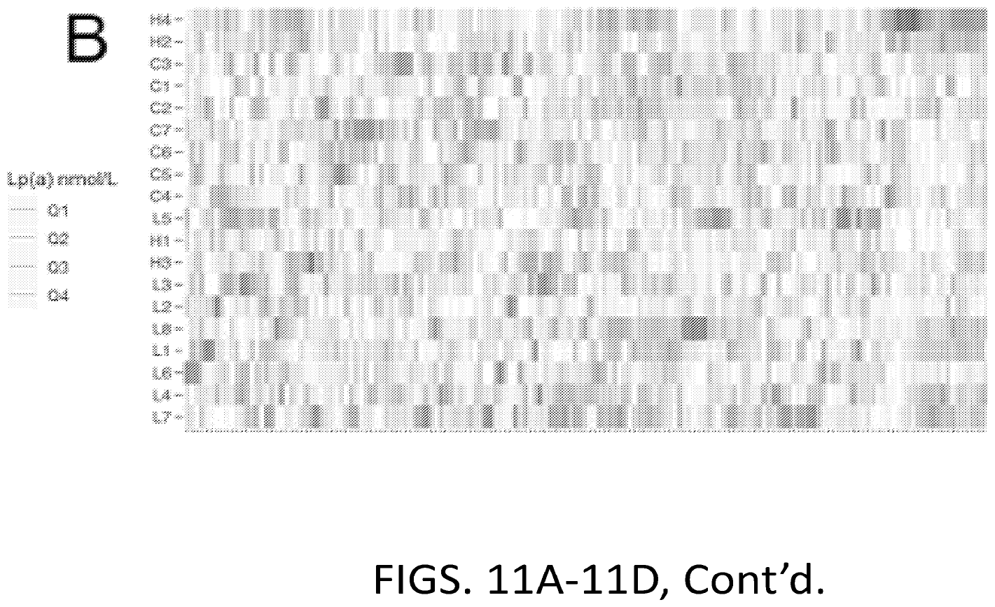

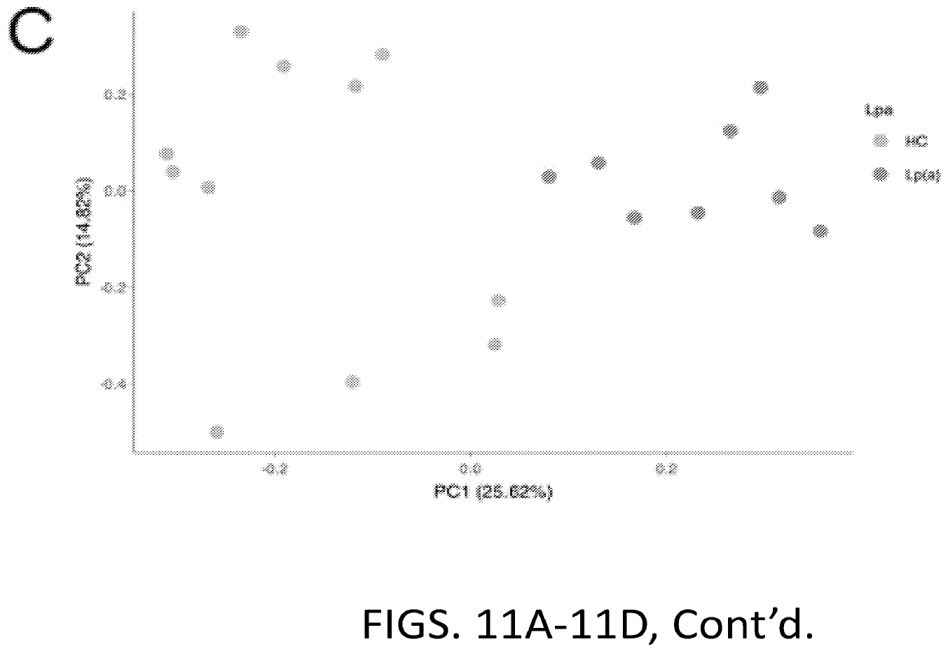

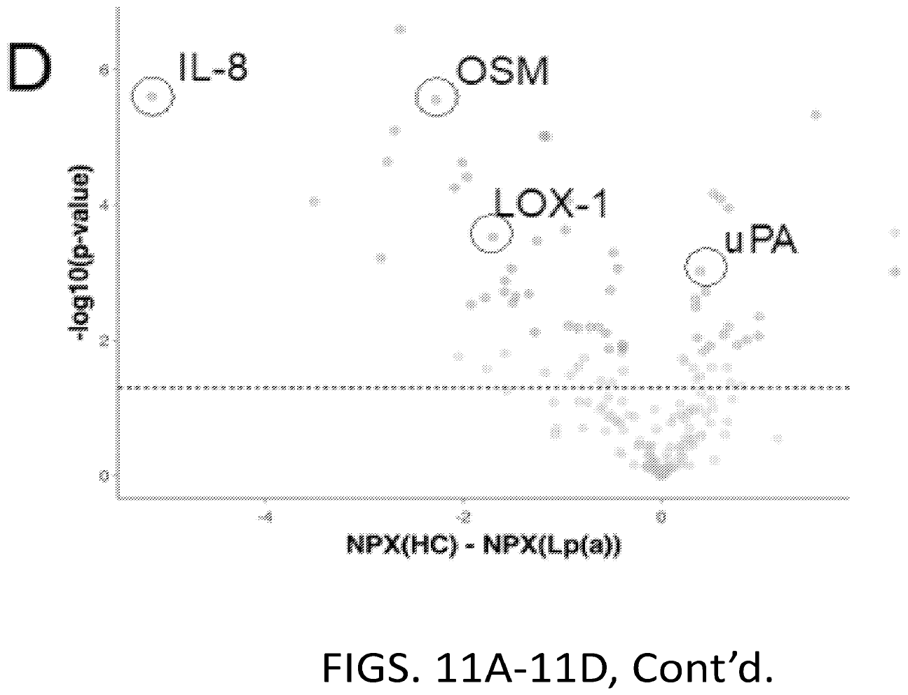

Several lines of evidence suggested that monocytes are the primary source of TF and interestingly, inflammatory mediators themselves can increase TF expression in circulating monocytes. To profile circulating monocytes, flow cytometry and a custom mass cytometry immune marker panel was used and circulating peripheral blood mononuclear cells (PBMCs) were profiled from CHD subjects. Results indicated that subjects with an elevated Lp(a) had increased classical monocytes and TF expression on CD14+ monocytes compared with age- matched controls. Subjects with an elevated vs. low Lp(a) had increased classical monocytes (15% vs 8%; p<0.05) (FIG. 11).

Results of the present disclosure also indicated that monocyte TF expression is increased by inflammatory stimuli. To determine if inflammatory mediators can drive monocyte TF expression, CD14+ monocytes were isolated from PBMCs and stimulated with TLR ligands (Pam2Csk4; Poly(I:C); LPS) for 4 hours. Using mass cytometry and qRT-PCR, results indicated that TLR ligands rapidly drive TF expression (both at the protein and transcriptional level) (FIG. 12).

TF is unique in that the presence of TF alone does not indicate TF activity. To assess if monocyte-bound TF is active, a FXa activity assay was employed. CD14+ monocytes were stimulated with LPS and FXa activity was assessed as a marker of TF activity. Results indicated that not only does LPS induce TF in monocytes, but it also increases TF activity as indicated by increased FXa activity (FIG. 12). Interestingly, when recognition of phosphatidylserine (PS), an apoptotic marker, is pharmacologically masked, TLR-driven TF expression is reduced, suggesting that parallel Mϕ sensing of TLR ligands and apoptotic cells/bodies drives immunothrombosis in CHD. These findings corroborated clinical observations that TF can be induced by inflammatory mediators and that monocytes themselves rapidly induce TF expression in response to TLR ligands and also support the hypothesis that immune-mediated induction of TF could drive the thrombosis associated with CHD.

Tissue factor activity is increased on circulating monocytes. Because of the striking increases in monocyte-TF upon exposure to inflammatory mediators and the increased circulating vascular/inflammatory mediators in CHD subjects with an elevated Lp(a), studies were conducted to determine if CHD subjects had evidence of increased TF/CD142 on circulating monocytes. To profile circulating immune cell populations, mass cytometry was used and age-matched controls were compared. Interestingly, CHD subjects with an elevated Lp(a) had increased classical monocytes (flow cytometry) and increased CD142/TF+ monocytes compared with age-matched controls. These results support findings from the plasma proteome in that even in presumed “stable” CHD subjects, elevated Lp(a) levels mark heightened inflammatory/vascular activation in the circulation.

Studies were also conducted to define the blood compartment distribution of TF in subjects with high Lp(a) concentrations. TF is found in 3 blood compartments: plasma (free TF), microvesicles, and circulating cells (primarily monocytes). It was hypothesized that TF is increased in all blood compartments in CHD subjects and that this increase is primarily driven by the Mϕ compartment. To define the blood compartment distribution of TF in CHD subjects, and how the TF compartment distributions in CHD subjects compares to distributions from age-matched controls, blood was separated in to 3 fractions (plasma, microvesicles, and circulating immune cells) and TF was measured using ELISA (BosterBio) and flow/mass cytometry (microvesicles/immune cells). Among circulating cells, monocytes express the majority of cell-bound TF. In patients with elevated Lp(a), it was found using mass cytometry that there is also an increase Mϕ TF expression. Peripheral activation of circulating monocytes has been demonstrated for inflammatory responses, but the effects of peripheral activation on Mϕ-mediated thrombosis remains largely unknown. The focus of this sub-aim is to use targeted proteomics and transcriptomics to define the mechanisms through which Lp(a) and inflammatory mediators (LPS [TLR4], ssRNA40 [TLR7/8]) drive Mϕ TF expression and activity.

These monocyte studies suggest that TLR ligands not only activate Mϕ inflammatory programs, but also induce TF. To explore the intersection of thrombosis and inflammation in Mϕ, PBMCs were isolated from subjects with CHD and age-matched controls and sorted on CD14 and CD142 expression. After isolating CD14+ and CD14+/CD142+ monocytes, monocytes were stimulated with the mediators described above. After stimulation, RNA and protein were isolated and processed for targeted transcriptomics (QuantSeq; Lexogen) and proteomics (microfluidic immunoblotting). Additionally, TF surface expression and TF activity were assessed in parallel.

ASCVD is associated with both heightened local/systemic inflammation and thrombotic potential. The innate arm of the immune response can drive both inflammation and thrombosis in ASCVD. Thrombosis triggered by the immune system, or immunothrombosis, is present in acute coronary syndrome (ACS) patients and those with unstable vascular lesions. Although pathologic in CHD, recent studies suggest that immunothrombosis is a physiologic pathogen response that works in concert with other effector arms of the innate immune system to acutely contain and eliminate the exogenous stresses. The immune system can activate coagulation through several procoagulant pathways. In the same way that unchecked inflammation can lead to tissue damage, dysfunction in immune-mediated coagulation can result in either pathologic thrombosis or coagulopathy, driving myocardial infarction, stroke, and disseminated intravascular coagulation (DIC) depending on the context.

Results of the present disclosure demonstrate differential transcriptional programs and signaling pathway activation that drive Mϕ TF expression in CHD subjects with high Lp(a) levels when compared to age-matched controls. Among subjects with high Lp(a) levels, higher concentrations of immune (chemokines/cytokines including IL-8 and TNFα) and vascular markers (P-selectin glycoprotein ligand-1 (PSGL-1), oncostatin M (OSM), urokinase plasminogen activator receptor (uPA)) were observed. These vascular makers bridge inflammation and thrombosis.

PSGL-1 is an immune checkpoint modulator that regulates signals of the of mobile cells of the immune system including macrophages/monocytes by selectin engagement as these cells migrate into and within the microenvironments in which they become localized. The signaling events regulate many facets of innate and adaptive immune responses. Leukocyte binding to the thrombi mediates the interaction of leukocyte PSGL-1 with P-selectin on the surface of activated platelets, and induces upregulation of leukocyte tissue factor, biosynthesis of several cytokines and other inflammatory reactions contributing to thrombotic progression. In contrast with PSGL-1, uPA reduces thrombosis. OSM is a gp 130 macrophage/T cell cytokine member of the IL-6 family that contributes to inflammation of the arterial wall and pathogenesis of atherosclerosis. It works synergistically with TLR-4 ligands to induce proinflammatory responses by arterial vascular smooth muscle cells and fibroblasts. Neutrophil released OSM enhances P-selectin mediated inflammation and thrombosis by promoting TF expression in vSMCs, a process mediated by through the activation of NFκB and that activation of NFκB is regulated in part by the MEK/Erk-1/2 signal transduction pathway.

A significant proportion of individual variation in immune responses is only identifiable after a stress or stimuli, reinforcing the notion that the immune system is inherently context specific. Lipopolysaccharide (LPS) induces human monocytes to express many proinflammatory mediators, including the procoagulant molecule tissue factor (TF) and the cytokine tumor necrosis factor alpha (TNF-alpha). The TF and TNF-alpha genes are regulated by various transcription factors, including nuclear factor (NFκB/Rel proteins and Egr-1. In this study, the role of the MEK-ERK1/2 mitogen-activated protein kinase (MAPK) pathway in LPS induction of TF and TNF-alpha gene expression in human monocytic cells.

Next, cell-based studies were conducted to identify transcriptional programs and signaling pathways. Results indicated that CAD patients with high Lp(a) levels have increased concentrations of classical monocytes demonstrating enhanced activation of inflammatory networks and immune-mediated activation of monocyte tissue factor expression and activity. To gain comprehensive insights into the mechanisms whereby Lp(a) mediates immune dysregulation and immunothrombosis, CyTof, differential transcriptional programs and signaling pathway activation that drive Mϕ TF expression, and functional assays of TF activity were used.

Thrombosis triggered by the immune system, or immunothrombosis, is associated with unstable coronary plaques and coronary thrombosis. TF is the molecular governor of the extrinsic coagulation pathway and is the key trigger of cell-mediated immunothrombosis. Stress-induced activation of TF works in concert with factor VII (FVII) to activate both factor X (FX) and factor IX (FIX), which then leads to thrombin generation and coagulation. In the absence of stress or infection, TF is not normally found in the circulation. Mechanistic studies have found that in response to pathogens, TF can be activated both in the vasculature and in circulating innate immune cells (primarily monocytes). Additionally, TF can be activated in endothelial cells by extracellular signals including vascular endothelial growth factor (VEGF), inflammatory cytokines including IL1b and TNFα, oxidized LDLs, and toll-like receptor (TLR) ligands. With respect to TLR ligands, Hirata and colleagues found that in vitro stimulation of THP-1 cells with LPS induced TF expression through NF-kB and AP-1 signaling. Additionally, previous studies have shown that LPS can also induce TF expression in HUVEC cells (endothelial cell model) and this induction was mediated by NF-kB signaling. TF can be activated both in the vasculature and in circulating innate immune cells (primarily monocytes). Understanding the mechanisms through which TF mediates both thrombotic sequalae and modulates immune responses in CHD has important implications in understanding the pathobiology of ASCVD and the drivers of CVD events in subjects with established CHD.

Using mass cytometry, results of the present disclosure indicated that there is also an increase Mϕ TF expression. Peripheral activation of circulating monocytes has been demonstrated for inflammatory responses, but the effects of peripheral activation on Mϕ-mediated thrombosis remains largely unknown. Profiling immune cell populations with mass cytometry afforded both breadth and depth; the optimized human immune panel allowed for parallel assessment of markers of cell identity and function (lineage, adhesion, migration, and both cell-surface/intracellular activation).

Understanding the mechanisms through which TF mediates both thrombotic sequalae and modulates immune responses in CHD has important implications in understanding the pathobiology of ASCVD and the drivers of CV events in subjects with established CHD. These unique approaches will not only inform understanding of the pathobiology of CHD but will also illuminate the conserved mechanisms that linking innate immune responses with pathologic thrombosis. Knowledge of these mechanisms has broad application to a wide spectrum of diseases including cancer, lung and other cardiovascular diseases.

### 4. Methods of Treatment

There is a natural arc to the induction and resolution of an immune response. In COVID-19, like other inflammatory conditions, the magnitude and duration of the immune response is orchestrated by an array of response elements with monocytes being central conductor of both inflammation and thrombosis. Overall, the results of the present disclosure link monocyte activation to both thrombosis and inflammation and how parenteral anticoagulation modulates the two in immunothrombotic conditions, which was previously unrecognized. The infrastructure of the ATTACC trial coupled with the incorporation of longitudinal immune profiling (biomarkers, cell populations, and ex vivo monocyte responses) provided the unique opportunity to define the mechanisms underlying immunothrombosis in COVID-19, and to define multi-dimensional immune signatures that could predict clinical outcomes for other immunothrombotic conditions. Importantly, understanding innate immune cell functional responses in parallel with markers of immune cell identity in immunothrombotic conditions will provide a more accurate snapshot of the host immune response and will discriminate which patients are containing the virus versus those who are not. More broadly, an improved understanding of the mechanisms mediating immunothrombosis could lead to novel disease-modifying therapies not only for COVID-19, but also for other diseases characterized by inflammation and thrombosis. In accordance with this, and as would be recognized by one of ordinary skill in the art based on the present disclosure, the compositions and methods provided herein can be applied to any disease indication mediated by TF, for both diagnostic and therapeutic purposes. For example, in addition to immunothrombotic conditions like cardiovascular diseases (e.g., ASCVD) and respiratory infections like COVID-19, the compositions and methods of the present disclosure can be used to identify and treat other disease indications, including but not limited to, rheumatoid arthritis (RA), inflammatory bowel diseases (e.g., Crohn's disease), chronic kidney disease, and any other acute and chronic inflammatory disorders

Thus, embodiments of the present disclosure include methods for treating a subject having or suspected of having an immunothrombotic condition. In some embodiments, the method includes obtaining a blood sample comprising a population of peripheral blood mononuclear cells (PBMCs) from a subject having an immunothrombotic condition, measuring a total level of TF and a level of TF activity in the sample obtained from a subject, and administering anti-thrombotic therapy and/or an apoptotic modulator to the subject to treat the immunothrombotic condition. In some embodiments, the method further comprises isolating the population of PBMCs from the blood sample. In some embodiments, measuring total TF levels comprises performing an immunoassay. In some embodiments, measuring total TF levels comprises performing a fluorometric assay. In some embodiments, measuring the TF activity level comprises measuring Factor Xa.

In some embodiments, the method further comprises obtaining a total TF level and a TF activity level in a sample obtained from a control subject. In some embodiments, the TF activity level is elevated in the sample from the subject having an immunothrombotic condition as compared to the TF activity level in the control. In some embodiments the immunothrombotic condition is selected from the group consisting of a virus infection, atherosclerotic cardiovascular disease (ASCVD), coronary heart disease (CHD), rheumatoid arthritis (RA), inflammatory bowel disease (IBD), chronic kidney disease, and any other acute and/or chronic inflammatory disorder. In some embodiments the virus infection is a SARS-CoV-2 infection.

In accordance with the above, methods of the present disclosure can include treatment with any medication and/or therapy that modulates one or more symptoms of an immunothrombotic condition. In some embodiments, treatment includes administering anti-thrombotic therapy to a subject based on the TF activity level, including but not limited to, administering a composition comprising heparin and/or Annexin V. The human vascular anticoagulant Annexin V is a 35-36 kDa, Ca²+-dependent phospholipid-binding protein that has a high affinity for the anionic phospholipid phosphatidylserine (PS). In normal healthy cells, PS is located on the cytoplasmic surface of the plasma membrane. However, during apoptosis, the plasma membrane undergoes structural changes that include translocation of PS from the inner to the outer leaflet (extracellular side) of the plasma membrane. It has been reported that the translocated phosphatidylserine on the outer surface of the cell marks the cell for recognition and phagocytosis by macrophages

In other embodiments, treatment includes administering an apoptotic modulator to a subject based on the TF activity level. In some embodiments, the apoptotic modulator induces apoptosis and treats the subject. In other embodiments, the apoptotic modulator reduces apoptosis and treats the subject. As would be recognized by one of ordinary skill in the art based on the present disclosure, apoptotic machinery can be activated by various intrinsic and extrinsic stimuli. The extrinsic pathway is activated by binding of specific ligands to so-called death receptors of the tumor necrosis factor (TNF) receptor superfamily at the cellular surface. Ligand binding to its cognate receptor (e.g., FasL-Fas/CD95, TRAIL-DR4 or DR5, TNF-TNFR1) leads to receptor trimerization and activation of intracellular death domains and recruitment of death domain-containing adapter proteins like Fas-associated death domain or TNF receptor-associated death domain that form a death-induced signaling complex which contains the proform of the initiator caspases 8 or 10. Extrinsic apoptosis induction is controlled at the level of signal transduction by inhibitory proteins like cellular FLICE-inhibitory protein and at the level of ligand binding by the expression of decoy receptors (DcR1, DcR2 and DcR3) lacking intracellular death domains. Apoptotic modulators can include, but are not limited to, monoclonal antibodies targeting death receptors and/or modulators of intracellular signaling cascades or protein turnover.

Embodiments of the present disclosure also include a kit comprising a TF detection agent, a TF activity detection agent, and instructions for performing an assay to determine a ratio of total TF to activated TF in a blood sample comprising a population of peripheral blood mononuclear cells (PBMCs). Embodiments of the present disclosure also include a kit comprising any of the compositions described herein to treat a subject with an immunothrombotic condition (e.g., identified as having elevated TF activity levels), and at least one container. In some embodiments, the kit further includes instructions for administering the composition to a human, including such information as dosing regimens, frequency of administration, routes of administration, side effects, and the like.

The various therapeutic/pharmaceutical compositions of the present disclosure can be provided to a subject with an immunothrombotic condition (e.g., identified as having elevated TF activity levels) in dosage forms, formulations, and in accordance with methods that confer advantages and/or beneficial pharmacokinetic profiles. A composition of the present disclosure can be utilized in dosage forms in pure or substantially pure form, in the form of its pharmaceutically acceptable salts, and also in other forms including anhydrous or hydrated forms. A beneficial pharmacokinetic profile may be obtained by administering a formulation or dosage form suitable for once, twice a day, or three times a day, or more administration comprising one or more composition of the present disclosure in an amount sufficient to provide the required concentration or dose of the composition to treat an immunothrombotic condition, as disclosed herein.

A subject may be treated with a composition of the present disclosure or composition or unit dosage thereof on substantially any desired schedule. They can be administered one or more times per day, in particular 1 or 2 times per day, once per week, once a month or continuously. However, a subject may be treated less frequently, such as every other day or once a week, or more frequently. A composition or composition may be administered to a subject for about or at least about 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously.

A beneficial pharmacokinetic profile can be obtained by the administration of a formulation or dosage form suitable for once, twice, or three times a day administration, or as often as needed, to treat an immunothrombotic condition. The required dose of a composition of the disclosure administered once twice, three times or more daily is about 0.01 to 3000 mg/kg, 0.01 to 2000 mg/kg, 0.5 to 2000 mg/kg, about 0.5 to 1000 mg/kg, 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 50 mg/kg, 0.1 to 20 mg/kg, 0.1 to 10 mg/kg, 0.1 to 6 mg/kg, 0.1 to 5 mg/kg, 0.1 to 3 mg/kg, 0.1 to 2 mg/kg, 0.1 to 1 mg/kg, 1 to 1000 mg/kg, 1 to 500 mg/kg, 1 to 400 mg/kg, 1 to 300 mg/kg, 1 to 200 mg/kg, 1 to 100 mg/kg, 1 to 50 mg/kg, 1 to 20 mg/kg, 1 to 10 mg/kg, 1 to 6 mg/kg, 1 to 5 mg/kg, or 1 to 3 mg/kg, or 1 to 2.5 mg/kg, or less than or about 10 mg/kg, 5 mg/kg, 2.5 mg/kg, 1 mg/kg, or 0.5 mg/kg twice daily or less.

The present disclosure also contemplates a formulation or dosage form comprising amounts of one or more compositions that results in therapeutically effective amounts of the composition over a dosing period, to treat an immunothrombotic condition. The therapeutically effective amounts of a composition of the disclosure are between about 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 75 mg/kg, 0.1 to 50 mg/kg, 0.1 to 25 mg/kg, 0.1 to 20 mg/kg, 0.1 to 15 mg/kg, 0.1 to 10 mg/kg, 0.1 to 9 mg/kg, 0.1 to 8 mg/kg, 0.1 to 7 mg/kg, 0.1 to 6 mg/kg, 0.1 to 5 mg/kg, 0.1 to 4 mg/kg, 0.1 to 3 mg/kg, 0.1 to 2 mg/kg, or 0.1 to 1 mg/kg.

A medicament or treatment of the disclosure may comprise a unit dosage of at least one composition of the disclosure to provide therapeutic effects. A “unit dosage or “dosage unit” refers to a unitary (e.g., a single dose), which is capable of being administered to a subject, and which may be readily handled and packed, remaining as a physically and chemically stable unit dose comprising either the active agents as such or a mixture with one or more solid or liquid pharmaceutical excipients, carriers, or vehicles.

### 5. Material and Methods

Enrolled patients. This study was approved by the Research and Development and Institutional Review Board committees at the LTC Charles S. Kettles VA Medical Center (Ann Arbor, MI). Informed consent was waived for the study. Plasma samples obtained at hospital admission for 12 symptomatic COVID-19 reverse transcription polymerase chain reaction (RT-PCR) confirmed cases that presented to the LTC Charles S. Kettles VA Medical Center were analyzed. Blood samples from patients with COVID-19 were drawn for clinical purposes and obtained from the hospital pathology laboratory following completion of clinical analysis, and were de-identified prior to analysis by the study laboratory team. Patient samples were stored at 4° C. for up to 48 hours prior to collection by the study team, then were frozen at −80° C. until analysis. Blood plasma was also collected from 7 healthy individuals (SARS-CoV-2 negative) who were used as controls. Control samples were acquired at the University of Illinois at Chicago prior the SARS-CoV-2 global outbreak from participants residing in the Greater Chicagoland area after providing written informed consent. All control samples were acquired after a standardized period (8-12 hrs) of overnight fasting.

PBMC treatment and stimulation. Approximately 10 million cells were equally divided into 4 fractions and treated with the following (for Hep experiments): 1) Hep (1.5 U/mL), 2) LPS (1 μg/mL), 3) LPS+Hep (1 μg/mL LPS with 1.5 U/mL Hep), and 4) RPMI medium alone. All treatments were performed for 2 hours in suspension at 37° C. and 5% CO₂ with gentle vortexing every 30 minutes to prevent cell sedimentation. After 2-hour incubation, cells were centrifuged, and supernatants were collected. Cells were then resuspended in 1 mL PBS (without Ca²⁺ and Mg ²⁺) for downstream analysis

RNA isolation and RNA-seq. Followed by treatments, bulk PBMC or CD14-positive PBMC were lysed with RLT PlusBuffer (QIAGEN, Cat. No. 1053393); RNA was purified with RNeasy Plus Micro Kit (QIAGEN, Cat. No. 74034) with additional DNA removal step by on-column digestion with RNase-Free DNase Set (QIAGEN, Cat. No. 79254) according to manufacturer's protocol. RNA-seq libraries were prepared using QuantSeq 3′ mRNA-Seq Library Prep Kit FWD for Illumina (Lexogen Cat. No. 015) and sequenced via Illumina NextSeq (HO) 150 cycle Sequencing by the Advanced Genomics Core at the University of Michigan. Sequencing data analysis and biological groups differential expression pairwise analysis was performed using QuantSeq data analysis pipeline provided by Lexogen and hosted on the BlueBee Genomics Platform. Gene Ontology, Pathway and Meta-Analysis of differentially expressed genes were performed using iPathwayGuide platform provided by Advaita Bioinformatics

PCR gene expression assay. RNA was then eluted in 32 uL RNase free water and concentration and purity were measured using Nanodrop. Applied Biosystems High-Capacity cDNA Reverse Transcription kit was used to convert RNA into cDNA for qPCR per the manufacturer's instructions. PCR was performed in 96 well plate using Taqman master mix and Taqman primers. 1× GAPDH-VIC and 1× target gene-FAM was used for each PCR reaction as housekeeping control gene and target gene respectively (total of 20 μL reaction volume). “No treatment control” is used to normalize each target gene expression for analysis of experimental groups.

Statistical analysis. Due to non-normality, data are presented as median (interquartile range), with between-group comparison using Mann-Whitney testing and p value <0.05 considered significant. The analyses were performed and depicted using GraphPad Prism (GraphPad Software, La Jolla, CA). Bivariate correlations were determined by use of Pearson correlation coefficient.

Study participants. Study participants were included with CHD who were aged 18 to 80 years with Lp(a) concentrations ≥150 nmol/L (cases) and <75 nmol/L (controls). Subjects with renal dysfunction (eGFR <3 mLmon/1.73 m²), history of active liver disease or hepatic dysfunction, active infection of autoimmune disease, treatment in the last 3 months with any of the following medications immunosuppressives, vitamin A derivatives and retinol derivatives for the treatment of dermatological conditions and niacin, were excluded. Subjects undergoing LDL apheresis, uncontrolled thyroid disease, cardiovascular event (acute coronary syndrome, PCI/CABG, stroke, peripheral arterial intervention) within 3 months, NYHA III or IV heart failure or last known ejection fraction <30%, type 1 diabetes or poorly controlled (HbA1c>8.5%) type 2 diabetes, were excluded.

Measurement of Lp(a) and oxPL:apoB. Lp(a) molar concentration in nmol/L was measured using a direct binding double monoclonal antibody-based ELISA reference method. Oxidized phospholipids on Lp(a) were measured by chemiluminescent assay (Boston Heart Labs, MA).

Profiling immune cell populations. Whole blood was obtained with Cytodelics stabilizer (for mass cytometry profiling), and CPT tubes (for functional profiling) as previously described. After isolating CD14+ and CD14+/CD142+ monocytes, monocytes were stimulated with the mediators described above. To minimize technical variability (processing, staining, and instrument), Pd CD45 barcoding was used, which allows for processing pooled and batched samples. For the mass cytometry profiling experiments, patient samples were collected and stored in tubes with stabilizing agent (Cytodelics; 2 mL). The antibody staining protocols for mass cytometry mirror that of flow cytometry. CyTOF1 mass cytometer (DVS/Fluidigm) was used for the data acquisition. PBMC were assessed after the exclusion of dead cells and doublets. The three main monocyte subsets of classical (˜85%), intermediate (˜5%), and non-classical (˜10%) monocytes were characterized by the level of CD14, CD16, and HLA-DR (classical: CD14^{High}CD16^{Low}; intermediate: CD14^{High}CD16^{High}; non-classical CD14^{Low}CD16^{High}).

Three algorithms/approaches (data visualization; stratifying; statistical analysis) were used to analyze CyTOF datasets. Analysis was done using a combination of X-shift, Citrus, and viSNE algorithms For de-barcoding and doublet removal, Boolean gating was used to deconvolute individual samples. All de-barcoded samples were converted into individual FCS files for further analysis. FCS files were compensated for signal spillover using R package CATALYST and transformed with ArcSinh transformation prior to analysis of surface and activation markers. X-shift was used as a data visualization tool and to monitor changes in immune cells and were compared against age-matched controls. Statistical significance was determined by one-factor ANOVA with Bonferroni correction and Student's t-test with p-value <0.05 considered significant.

After stimulation, RNA and protein was isolated and processed for targeted transcriptomics (QuantSeq; Lexogen) and proteomics (microfluidic immunoblotting) as described. The plasma proteome was defined using the Olink proteomics. Olink proteomic reagents are based on Proximity Extension Assay (PEA) technology, where oligonucleotide labeled antibody probe pairs bind to respective target proteins. The proteomic reagents are based on Proximity Extension Assay (PEA) technology, where oligonucleotide labeled antibody probe pairs bind to respective target proteins in plasma/serum. A PCR reporter sequence is formed by a proximity-dependent DNA polymerization; this sequence is amplified and quantified using real-time PCR. To complement the proteomic profiling described above, the Bio-Plex Pro Human Cytokine Screening Panel (Bio-Rad; CA) was used to interrogate 48 inflammatory mediators. Quantifying cytokines in tandem with circulating immune cells allowed for the categorization of immune trajectories to type 1/2/3 immune responses and how these signatures relate to Lp(a) levels and clinical variables.

Measurement of tissue factor mediated thrombosis. TF surface expression and TF activity were assessed in parallel with microfluidic immunoblotting experiments. ELISA (presence or absence of TF), flow/mass cytometry (Mϕ bound TF), fluorogenic TF assays (TF activity), and fibrin-clot assays (physiologic assessment of clot formation) were used to interrogate multiple dimensions of TF-mediated thrombosis in CHD patients. The blood was separated in to 3 fractions (plasma, microvesicles, and circulating immune cells) and TF was measured using ELISA (BosterBio) and flow/mass cytometry (microvesicles/immune cells). Because microvesicles lack a cell nucleus which is an important discriminator employed in mass cytometry, TF expression on microvesicles was profiled using flow cytometry. In brief, microvesicles were isolated using ultracentrifugation (UC) coupled with separation (sucrose gradient), PKH67 labeling, and staining with primary antibodies, and flow cytometric analysis as previously described.

Active TF catalyzes the conversion of factor X to Xa; TF activity in plasma, microvesicles, and monocytes were measured by quantifying factor Xa activity using the fluorogenic factor Xa substrate (Abcam). TF activity was quantified based on the ability of TF/FVIIa to activate FX to FXa. The amidolytic activity of the TF/FVIIa complex was quantitated by the amount of FXa produced using a FXa substrate which releases a chromophore upon cleavage, and which can be detected by spectrophotometry. To confirm TF activity specificity, anti-TF antibodies (Abcam) and TFPI (Sigma) was added to blood components to block TF activity.

Results from the fluorogenic TF assay were normalized to total TF levels, which provided a normalized factor Xa activity level that accounts for differences in total TF levels. In addition, experiments were performed with the anti-TF antibodies and TFPI over a range of concentrations and IC50 for each was determined. These complementary approaches allow for the separation of TF-mediated thrombosis secondary to increasing amounts of TF from that of increasing TF activation.

A viSNE-based algorithm was used to examine changes in TF surface expression on circulating immune cells and Citrus-based algorithm to determine differences in TF expressing cell populations in CHD subjects with and without Alirocumab compared with age-matched controls. Statistical significance was determined by one-factor ANOVA with Bonferroni correction and Student's t-test with p-value <0.05 considered statistically significant. GraphPad, Prism Inc., was used for statistical analysis. Unpaired t-test and ANOVA based algorithms were used to determine significance between CHD subjects and controls for both TF cell population differences, circulating TF levels, and TF activity.

Tests of association between mixed continuous versus non-ordered categorical variables were performed by unpaired Wilcoxon test (for n=2 categories) or by Kruskal-Wallis test (for n>2 categories). Association between categorical variables were assessed by Fisher-exact test. Unsupervised cluster analysis was performed on baseline and day 3 phenotyping variables. All tests were performed two-sided, using a nominal significance threshold of P<0.05. When appropriate to adjust for multiple hypothesis testing, false discovery rate (FDR) correction were performed using the Benjamini-Hochberg procedure at the FDR<0.05 significance threshold Immunoblots were analyzed using ImageJ software as previously described. Gene expression was analyzed using comparative Ct method (ΔΔCt) and normalized to GAPDH.

Measurement of inflammatory mediated TF expression. To determine if inflammatory mediators can drive monocyte TF expression, CD14+ monocytes were isolated from peripheral blood mononuclear cells (PBMC) and were stimulated with TLR ligands (Pam2Csk4; Poly(I:C); LPS) for 4 hours. To assess if monocyte-bound TF is active, a FXa activity assay was used. CD14+ monocytes were stimulated with LPS and FXa activity was assessed as a marker of TF activity.

What is claimed is:

1. A method comprising:
(a) obtaining a blood sample comprising a population of peripheral blood mononuclear cells (PBMCs) from a subject having an immunothrombotic condition; and
(b) measuring a total level of Tissue Factor (TF) and a level of TF activity in the sample obtained from the subject.

2. The method according to claim 1, wherein the method further comprises isolating the population of PBMCs from the blood sample.

3. The method according to claim 1 or claim 2, wherein the method further comprises isolating a population of monocytes and/or macrophages from the PBMCs.

4. The method according to claim 3, wherein the method comprises measuring the total TF level and the TF activity level from the population of monocytes and/or macrophages.

5. The method of according to any of claims 1 to 4, wherein measuring total TF levels comprises performing an immunoassay.

6. The method according to any of claims 1 to 4, wherein measuring total TF levels comprises performing a fluorometric assay.

7. The method according to any of claims 1 to 6, wherein measuring the TF activity level comprises measuring Factor Xa.

8. The method according to any of claims 1 to 7, wherein the method further comprises determining a ratio of total TF levels to TF activity levels.

9. The method according to any of claims 1 to 7, wherein the method further comprises obtaining a total TF level and a TF activity level in a sample obtained from a control subject.

10. The method according to any of claims 1 to 7, wherein the method further comprises measuring a total TF level and a TF activity level in a sample obtained from a control subject.

11. The method according to any of claims 1 to 10, wherein the TF activity level is elevated in the sample from the subject having an immunothrombotic condition as compared to the TF activity level in the control.

12. The method according to any of claims 1 to 11, wherein the immunothrombotic condition is selected from the group consisting of a virus infection, atherosclerotic cardiovascular disease (ASCVD), coronary heart disease (CHD), rheumatoid arthritis (RA), inflammatory bowel disease (IBD), chronic kidney disease, and any other acute and/or chronic inflammatory disorder.

13. The method according to claim 12, wherein the virus infection is a SARS-CoV-2 infection.

14. The method according to claim 12, wherein the IBD is Crohn's disease.

15. The method according to any of claims 1 to 14, wherein the immunothrombotic condition is characterized by an altered level of at least one biomarker.

16. The method according to claim 15, wherein the at least one biomarker comprises hyaluronan (Hyal), syndecan-1 (SDC1), interleukin 6 (IL-6), tumor necrosis factor alpha (TNFα), Lipoprotein(a) (Lp(a)), interleukin 8 (IL-8), P-selectin glycoprotein ligand-1 (PSGL-1), and oncostatin M (OSM), heparan sulfate (HS), high-sensitivity cardiac troponin hs-cTn), high-sensitivity C-reactive protein (hs-CRP), low-density lipoprotein (LDL), von Willebrand factor (vWF), and any combinations thereof.

17. The method according to any of claims 1 to 16, wherein the method further comprises measuring a level of at least one biomarker.

18. The method according to claim 17, wherein the at least one biomarker comprises hyaluronan (Hyal), syndecan-1 (SDC1), interleukin 6 (IL-6), tumor necrosis factor alpha (TNFα), Lipoprotein(a) (Lp(a)), interleukin 8 (IL-8), P-selectin glycoprotein ligand-1 (PSGL-1), and oncostatin M (OSM), and any combinations thereof.

19. The method according to claim 17, wherein the at least one biomarker is Lp(a), and wherein the Lp(a) is elevated in the sample from the subject having an immunothrombotic condition as compared to the Lp(a) level in the control.

20. The method according to claim 17, wherein the at least one biomarker is IL-6, and wherein the IL-6 is elevated in the sample from the subject having an immunothrombotic condition as compared to the IL-6 level in the control.

21. The method according to claim 17, wherein the at least one biomarker is selected from the group consisting of RAGE, CD40, CCL25, CXCL6, TNFα, CXCL5, PD-L1, MMP1, IL-18, CXCL1, Trail, OSM, uPA, IL-7, IL-8, Dkk-1, CCL17, IL-18, LOX1, CXCL1, PAR1, Angpt1, and CD40L; and
wherein the at least one biomarker is altered in the sample from the subject having an immunothrombotic condition as compared to the level in the control.

22. The method according to any of claims 1 to 21, wherein the method further comprises treating the subject based on the TF activity level.

23. The method according to claim 22, wherein treating the subject comprises administering an anti-thrombotic therapy.

24. The method according to claim 23, wherein the anti-thrombotic therapy comprises administering a composition comprising heparin and/or Annexin V.

25. The method according to claim 22, wherein treating the subject comprises administering an apoptotic modulator.

26. The method according to claim 25, wherein the apoptotic modulator induces apoptosis and treats the subject.

27. The method according to claim 25, wherein the apoptotic modulator reduces apoptosis and treats the subject.

28. A method for treating a subject having or suspected of having an immunothrombotic condition, the method comprising:
(a) obtaining a blood sample comprising a population of peripheral blood mononuclear cells (PBMCs) from a subject having an immunothrombotic condition;
(b) measuring a total level of Tissue Factor (TF) and a level of TF activity in the sample obtained from a subject; and
(c) administering anti-thrombotic therapy and/or an apoptotic modulator to the subject to treat the immunothrombotic condition.

29. The method according to claim 28, wherein the method further comprises isolating the population of PBMCs from the blood sample.

30. The method of according to claim 28 or 29, wherein measuring total TF levels comprises performing an immunoassay.

31. The method according to claim 28 or 29, wherein measuring total TF levels comprises performing a fluorometric assay.

32. The method according to any of claims 28 to 31, wherein measuring the TF activity level comprises measuring Factor Xa.

33. The method according to any of claims 28 to 32, wherein the method further comprises obtaining a total TF level and a TF activity level in a sample obtained from a control subject.

34. The method according to any of claims 28 to 33, wherein the TF activity level is elevated in the sample from the subject having an immunothrombotic condition as compared to the TF activity level in the control.

35. The method according to any of claims 28 to 34, wherein the immunothrombotic condition is selected from the group consisting of a virus infection, atherosclerotic cardiovascular disease (ASCVD), coronary heart disease (CHD), rheumatoid arthritis (RA), inflammatory bowel disease (IBD), chronic kidney disease, and any other acute and/or chronic inflammatory disorder.

36. The method according to claim 35, wherein the virus infection is a SARS-CoV-2 infection.

37. The method according to any of claims 28 to 36, wherein the anti-thrombotic therapy and/or the apoptotic modulator is administered to the subject based on the TF activity level.

38. The method according to claim 37, wherein the anti-thrombotic therapy comprises administering a composition comprising heparin and/or Annexin V.

39. The method according to claim 37, wherein the apoptotic modulator induces apoptosis and treats the subject.

40. The method according to claim 37, wherein the apoptotic modulator reduces apoptosis and treats the subject.

41. A kit comprising:
a Tissue Factor (TF) detection agent;
a TF activity detection agent; and
instructions for performing an assay to determine a ratio of total TF to activated TF in a blood sample comprising a population of peripheral blood mononuclear cells (PBMCs).
